(19) United States
(12) Patent Application Publication
(10) Pub. No.: US 2011/0152600 A1
(43) Pub. Date: Jun. 23, 2011

(54) IMPLANTABLE HEART ASSIST SYSTEM

(75) Inventors: Michael Scott, Lake Forest, CA (US); Rex O. Bare, Lake Forest, CA (US); Robert Miller, Costa Mesa, CA (US); Jeffrey Smith, Costa Mesa, CA (US); Andrew Moulds, Encinitas, CA (US)

(73) Assignee: Thoratec Corporation, Pleasanton, CA (US)

(21) Appl. No.: 13/061,086

(22) PCT Filed: Aug. 28, 2009
(86) PCT No.: PCT/US09/55431

§ 371 Date: Feb. 25, 2011

Related U.S. Application Data
(60) Provisional application No. 61/092,714, filed on Aug. 28, 2008.

(51) Int. Cl. A61M 1/10; A61N 1/08

(52) U.S. Cl. 600/16; 607/60

(57) ABSTRACT

A heart assist system having an implantable pump conveying blood between two vascular locations and an extracorporeal system providing power and control signals to the pump. The system also includes a communication link having an implantable portion coupled to the implantable pump, an extracorporeal portion coupled to the extracorporeal system and an isolation portion between the implantable portion and the extracorporeal portion that minimizes the transmission of movement and forces from the extracorporeal portion to the implantable portion.

### RELATED APPLICATIONS

This application claims priority to U.S. Provisional Application Ser. No. 61/092,714 filed Aug. 28, 2008 entitled Implantable Heart Assist System, which is hereby incorporated herein by reference. Also incorporated herein by reference is U.S. application Ser. No. 11/694,761 filed Mar. 30, 2007.

### FIELD OF THE INVENTION

The present invention relates to a heart assist system and particularly to an implantable heart assist system.

### BACKGROUND OF THE INVENTION

Heart disease is a growing epidemic in the United States that can lead to heart failure. Heart disease is a progressive, chronic disease with total mortality in 2002 approaching 300,000. AHA 2007 Heart Disease & Stroke Statistics. In the United States alone, 5.2 million people have congestive heart failure with more than one million hospitalizations and 550,000 new diagnosis annually. Id. The total cost of heart failure in the United States is more than $33 billion. Also, US hospital costs for heart failure exceed $15 billion, more than 50% of total costs.

Heart failure is characterized as a progressive, downward spiral. In particular, cardiac injury can cause cardiac dysfunction, which results in reduced cardiac output. One result of reduced cardiac output is endothelial dysfunction, neurohormonal activation, renal impairment, and vasoconstriction. These results can lead to fluid retention and increased systemic vascular resistance. An increase in systemic vascular resistance can create increased cardiac load which can cause further cardiac dysfunction. Thus a cycle of further cardiac dysfunction can be established.

Although there are various treatments proposed and being developed for treating heart failure, such systems are generally limited to the hospital setting or at least require the patient to be very limited in mobility if not completely confined to a bed.

### OBJECTS AND SUMMARY OF THE INVENTION

The present invention relates to a system for assisting a patient's heart and, in particular, to a system that can enable a patient to be ambulatory during treatment. Various embodiments discussed herein are related to implantable heart assist systems and methods for augmenting flow within the vasculature of the patient. Additional features of heart assist systems that can be combined with the features described herein are set forth below.

In one embodiment, a heart assist system is provided that includes an implantable pump, an extracorporeal system, and a communication link. The implantable pump is configured to convey blood between two vascular locations. The extracorporeal system provides power and control signals to the pump. The communication link is coupled with the extracorporeal system and with the pump for conveying information therebetween. The communication link can also convey power to the pump. The communication link comprising includes an implantable portion having a distal end configured to couple with the implantable pump and an extracorporeal portion having a proximal portion configured to couple with the extracorporeal system. Also, the communication link has an isolation portion disposed between the implantable portion and the extracorporeal portion. The isolation portion is configured to minimize the transmission of at least one of movement of and forces from the extracorporeal portion to the implantable portion.

The isolation portion can be any structure that can be lengthened or change its natural shape to absorb movements or forces that would otherwise be conveyed to a percutaneous site (e.g., a skin puncture through which the communication link extends). For example, a spiral portion can be coiled and uncoiled in response to movement and forces without disrupting the percutaneous site.

In one embodiment, a communication link is provided for conveying signals between a extracorporeal controller and an implantable pump. The communication link includes a distal end, a proximal end, and an elongate body extending therebetween. The elongate body has a plurality of lumens that extend therethrough. A signal wire extends through each of the lumens. The signal wires convey at least one of power and control signals to the pump. The signal wires also can convey data to the controller. A plurality of contacts is located at the proximal end for placing the communications link in electrical connection with the controller. A plurality of contacts is located at the distal end for connecting the communications link with the pump. The lumens can have a helical arrangement relative to each other to reduce electrical noise and to reduce stress on the wires.

An apparatus is provided, in another embodiment, for disconnectably connecting a percutaneous signal line to a signal source. The apparatus includes a first connector portion coupled with the percutaneous signal line and a second connector portion electrically coupled with a signal source for conveying control signals between the signal source and the percutaneous signal line. The first connector portion also includes a housing having a distal end and a proximal end. The proximal end has a recess formed therein. The recess comprises a first ramped surface and a second ramped surface positioned distal of the first ramped surface. The second connector portion has a protruding portion extendable into the recess along a connection axis and a compressible member coupled with the protruding portion. The compressible portion extends away from the connection axis by a first amount in the absence of external forces. Distal advancement of the protruding portion in the recess along the connection axis causes the compressible portion to be brought into engagement with the first ramped surface. Further distal advancement of the protruding portion in the recess along the connection axis causes the compressible member to be compressed toward the protruding portion. Still further distal advancement of the protruding portion in the recess along the connection axis causes the compressible member to expand along the length of the second ramped portion.

In another embodiment, an apparatus is provided for disconnectably connecting a percutaneous signal line to a signal source. The apparatus includes a first connector portion and a second connector portion. The first connector portion is coupled with the percutaneous signal line and has a housing that has a distal end and a proximal end. The proximal end has a recess formed therein. The second connector portion is electrically coupled with a signal source for conveying control signals between the signal source and the percutaneous signal line. The second connector portion has a protruding portion extendable into the recess along a connection axis. The first and second connector portions can be connected by a force that is substantially less than a force required to disconnect the first and second connectors.

The embodiments for disconnectably connecting components can be reversed such that a protruding portion is provided on the percutaneous signal line and a recess can be formed on a separable component, such as a patient lead or controller signal line.

In another embodiment, an apparatus for drawing a percutaneous conduit through a tissue tunnel is provided. The apparatus includes a forward portion having a tissue displacing surface and a rearward portion engageable with a proximal end of a percutaneous conduit. A seal is provided that is configured to engage an inside surface of the proximal end of the percutaneous conduit to prevent ingress of bodily tissue and fluid from the tunnel into the proximal end of the percutaneous conduit. The apparatus also includes a tension member for transmitting a pulling force from a proximal end of a tunnel, through the tunnel to at least one of the forward and rearward portions and to the percutaneous conduit.

In another embodiment, a tunneling apparatus is provided for pulling a conduit through subcutaneous tissue. The tunneling apparatus includes a tissue displacing surface disposed on a forward portion thereof, a tension member, and a securement mechanism. The tension member is configured to pull the tissue displacing surface into engagement with tissue surrounding the tunnel in front of the tissue displacing surface. The securement mechanism is disposed rearwardly of the tissue contacting surface. The securement mechanism is configured to mechanically couple an end portion of the conduit with the tunneling apparatus.

Various methods also can be provided. For example, in one embodiment, a method of applying a percutaneous heart support system is provided. In this method, a subcutaneous pocket is formed in the patient. A pump is positioned in the subcutaneous pocket. A tension member of a tunneling assembly is moved through subcutaneous tissue and through a percutaneous site spaced apart from the subcutaneous pocket. A tunneling body is coupled to a proximal end of a percutaneous conduit. The percutaneous conduit is coupled with the pump. A tension force is applied to the tension member to draw the proximal end of the percutaneous conduit proximally through subcutaneous tissue to the percutaneous site.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which

FIG. 1 is an exploded view of one embodiment of an implantable system for augmenting bloodflow in a patient;

FIG. 2 is a schematic view of one embodiment of an implantable bloodflow system, shown applied to a patient's vascular system;

FIG. 3 is a plan view of a pump assembly;

FIG. 3A is a plan view of a portion of the pump assembly illustrating one embodiment of a percutaneous interface portion;

FIG. 3B shows detail 3B-3B illustrated in FIG. 3A;

FIG. 4 is an exploded view of a pump header assembly;

FIG. 5 is a perspective view of the pump assembly of FIG. 3, illustrating an isolation portion;

FIG. 5A shows the detail 5A-5A including an electrical connection between a communication link and an implantable pump;

FIG. 6 is an exploded view of the pump assembly of FIG. 3 illustrating a connector for coupling the pump with a system controller;

FIG. 6A illustrates the detail 6A-6A shown in FIG. 6, illustrating features of the connector including contacts and signal wires;

FIG. 7 illustrates a portion of a header assembly coupled with the implantable pump of FIG. 3;

FIG. 8 is a detail view of an electrical connection formed at a header assembly between one or more signal wires and corresponding post connector(s) of an implantable pump;

FIG. 9 is a detailed view of one embodiment of an isolation portion configured to protect a percutaneous site;

FIG. 10 is a plan view of one embodiment of a keyed socket or connection portion;

FIG. 11 is a cross-sectional view of the keyed socket of FIG. 10 taken along section plane 10-10;

FIG. 12 is a cross-sectional view of the keyed socket of FIG. 10 taken along section plane 12-12;

FIG. 13 is a perspective view of a keyed plug configured to couple with the keyed socket of FIG. 10;

FIG. 14 illustrates steps of one method for implanting a pump assembly, including an implantable pump and a percutaneous communications link;

FIG. 15 is a plan view of a tunneling apparatus configured to couple with a percutaneous conduit;

FIG. 16 is a cross-section of the tunneling apparatus shown in FIG. 15 taken along section plane 16-16;

FIG. 17 in the plan view of a leading portion of the tunneling apparatus of FIG. 15;

FIG. 18 is a cross-sectional view of the leading portion of FIG. 17, taken along section plane 18-18;

FIG. 19 is a perspective view of a trailing portion of the tunneling apparatus of FIG. 15;

FIG. 19A is a plan view of the trailing portion of the tunneling apparatus of FIG. 15;

FIG. 19B is a cross-section view of the trailing portion taken along section plan 19B-19B in FIG. 19A;

FIG. 20 is a perspective view of an anchor of the tunneling apparatus of FIG. 15.

### DESCRIPTION OF EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

This application is directed to implantable apparatuses, systems, and methods for treating cardiovascular insufficiency, e.g., heart disease, congestive heart failure, and related conditions and symptoms. The apparatuses and systems described herein can be used to treat chronic conditions and preferably enable the patient to be ambulatory, such that the patient is able to conduct many of the normal activities of a healthy person. Accordingly, the apparatuses and systems described herein are configured to be robust in an ambulatory mode.

The systems and apparatuses can be deployed to minimize further reduction in or improve cardiac output. In some cases, the apparatuses, systems, and methods described herein can be deployed to reduce or prevent further increases in cardiac load due to a cardiovascular condition. Such apparatuses, systems, and methods can be deployed to remedy one or more of endothelial dysfunction, neurohormonal activation, renal impairment, and vasoconstriction. Furthermore, such apparatuses, systems, and methods can be deployed to decrease or minimize an increase in fluid retention or systemic vascular resistance due to a cardiovascular condition. Some apparatuses and systems described herein are well suited for modifying a flow regime in a patient's aorta or other vascular portion, for example by reducing or eliminating disordered flow in the aorta or other vascular portion.

Other related methods and apparatuses involve techniques and devices for constructing or for applying such apparatuses and systems, e.g., for implanting at least a portion of the system or apparatus within the patient.

I. Implantable Extracardiac Heart Assist Systems and Methods

Many of the apparatuses and systems described herein are intended to allow the patient to be ambulatory, for example by configuring components to be implantable. In some embodiments, at least a portion of the apparatus or system is configured to be disposed outside the patient, e.g., having a low profile configuration. In some apparatuses and systems a percutaneous structure is provided that extends between implantable components and extracorporeal components. Various advantageous features are discussed below that help to protect or sustain the viability of percutaneous structures and percutaneous sites though which they extend.

A. System Overview

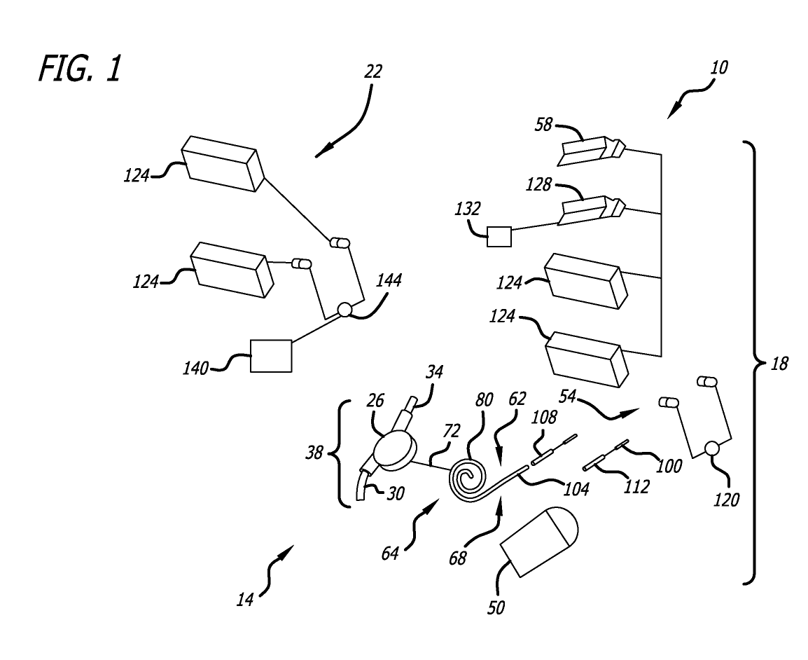

FIG. 1 is an exploded view of one embodiment of a circulation supplementing system 10. The system 10 includes an implantable portion 14, an extracorporeal portion 18 and a power management system 22. In some applications, the implantable portion 14 and the extracorporeal portion 18 provide an ambulatory treatment system. For example, the extracorporeal portion 18 can be configured to be worn by the patient. As such, the term “wearable portion” is sometime used herein to describe such an apparatus and application. The auxiliary power system 22 enables the patient to maintain continuous power to electrical components of the system 10.

In one embodiment, the implantable portion 14 includes a pump 26, an outflow blood conduit 30, and an inflow blood conduit 34. The outflow blood conduit 30, the inflow blood conduit 34, and blood contacting portions of the pump 26 define portions of a bloodflow circuit 38 through which blood is conveyed to augment flow in a selected region of the vasculature. In one mode of operation blood is drawn into the inflow blood conduit 34 by the pump 26 and is delivered through the outflow blood conduit 30 into the patient's vasculature. In one treatment, blood is delivered into the vasculature from the outflow blood conduit 30 in a manner that enables the system 10 to augment flow within the vasculature.

In some applications, continuous flow augmentation is provided as a treatment for decompensated heart failure. For example, a continuous flow component can be directed to a selected region of the aorta to enhance the otherwise pulsatile flow in that region to enhance flow in that region. Continuous full augmentation, e.g., in the aorta, is one way to overcome disordered blood flow. For example, continuous aortic flow augmentation can reorder aortic flow. Suitably ordered flow can improve endothelial function in some applications. Also, in some modes the systems described herein can reduce neurohormonal down regulation. In some applications, the system 10 and related systems can improve renal vasodilation, whereby more oxygenated blood can reach the kidneys. As a result, fluid removal from the blood can be improved to reduce systemic vascular resistance. This can lead to a decrease in cardiac load. These benefits can lead to improved cardiac function, as discussed further below.

The extracorporeal portion 18 can include a system controller 50, a power supply 54, and a data link 58. In some embodiments, one or more of the system controller 50, the power supply 54, and the data link 58 is configured to be low-profile such that the patient can wear these components close to their body. Also, in some embodiments, one or more of the system controller 50, the power supply 54 (e.g., providing AC mains power), and the data link 58 is configured to be light-weight so that the patient can be ambulatory with relative comfort. In addition to the power supply 54, one or more primary batteries and secondary batteries 124 (e.g., rechargeable batteries) can be incorporated into the system 10. Also, a DC power supply (e.g. an adapter for a car, plane or other vehicle) can be coupled with the system 10. In some embodiments, a power supply can include the capability of being powered by conventional commercial batteries, such as D-cell batteries. Such configurations of these components of the system 10 enable the patient to carry on many of the activities of healthy person.

In one embodiment, the controller 50 is capable of wireless communication, e.g., using Bluetooth or another wireless protocol, with a computer or other data analysis system. Where the controller 50 is capable of wireless communication, the data link 58 can be provided as a redundant communication device should the wireless connection fail. In one embodiment, the system controller 50 includes a power source (e.g., a backup battery) that can operate the system 10 independent of externally-supplied power for a short time. In one power management protocol, the controller 50 can sense or otherwise take note of a lack of external power and respond by implementing a power conservation mode. In one implementation a power conservation mode causes the controller 50 to begin to draw power from an available power source, such as an internal backup battery, and reduce the RPM of the pump 26 to minimize power consumption to maximize runtime. This power conservation mode can give the patient more time for to connect to external power.

In some embodiments, a communication fink 62 extends between the system controller 50 and the pump 26. The communication link 62 can take any suitable form. In various embodiments discussed in greater detail below, the communication link 62 provides different advantageous features to implantable systems. For example, as discussed below, the communication link 62 can be configured to minimize the effect of external forces acting on and movements or perturbations of the extracorporeal portion 18 on the implantable portion 14 of the system 10. Also, in some embodiments, the communication link 62 and its manner of connection to the system controller 50 are configured to disconnect at a force that is lower than a threshold force above which portions of the implantable portion 14 would become disrupted. For example, as discussed further below, an implantable length of the communication link 62 is configured to enhance tissue in-growth with the surrounding tissue at a percutaneous site and, in some embodiments, along a subcutaneous length of the communication link 62. A connection located between the communication link 62 and the system controller 50 can be configured to disconnect under a force that, if transmitted to the percutaneous site, could damage such in-growth.

In some embodiments, a connection between the communication link 62 and the pump 26 is configured to be able to manage relative movement between these components. As discussed further below, one feature of the communications link 62 is the minimization of movement of a portion thereof that is subcutaneous or at the skin exit site relative to the patient's tissue. This feature minimizes relatively small movements that will prevent tissue in-growth into a tissue in-growth material or structure, discussed below. Disruption of in-growth can prevent healing, which in turn can prevent a biological barrier from developing. Such a barrier is advantageous in that it can prevent infectious agents from migrating along the communications link 62 to a warm, moist location adjacent to healing tissue where these agents may cause infection. By isolating movement from the exit site, healing is encouraged more rapidly and completely.

Also, movement of portions of the implantable portion 14 can be significant because, as discussed further below, the system 10 is intended to be implanted near the patient's waist, which is a location that undergoes significant bending and jostling. These and other body movements result in fatigue cycles that could cause breakage of the mechanical or the electrical connections between the communication link 62 and the pump 26. A break-down in the mechanical connection can be problematic because it can leave the electrical connections exposed to the body cavity, which could lead to a break-down in the electrical circuit. Also, a break-down in the mechanical connection could lead to generation of loose matter in the patient's body, which could lead to infection, irritation, and potentially a need to explant the device. A break-down in the electrical connection would likely prevent control signals or power from the controller 50 from reaching the pump 26. In either event, the pump 50 will not operate as intended and may in fact stop pumping altogether. A lengthy period of non-operation by the pump 50 would require costly and inconvenient intervention by a clinician, and, perhaps, explant.

In one embodiment the communication link 62 includes percutaneous conduit 64 that includes an extra-corporeal portion 68 and a subcutaneous portion 72. The subcutaneous portion 72 includes a first end configured to couple with the pump 26 in a robust manner such that the subcutaneous portion 72 and the pump 26 will not become disconnected inadvertently during normal use. The subcutaneous portion 72 also includes an elongate portion that is configured to reside within the patient in a biocompatible manner, for example, being integrated into the surrounding tissue. Such integration can be achieved by tissue in-growth, as discussed further below. Tissue in-growth and other approaches to integration into the surrounding tissue are advantageous in minimizing a potential for infection, e.g., by entry of bacteria into the patient through a percutaneous site and, potentially, along the outer wall of the subcutaneous portion 72.

Although the subcutaneous portion 72 can be configured to integrate with the tissue (e.g. by in-growth), other sections may be configured to discourage in-growth. For example, portions distant from the exit site, such as the pump 26 and distal-most portions of the communications link 62 can be configured to discourage such integration. This is advantageous in that discouraging integration can minimize adverse effects, such as fibrosis and the build-up of excessive scar tissue which could make servicing or replacing these components more difficult. In some embodiments, subcutaneous portions are configured to discourage in-growth by being smooth.

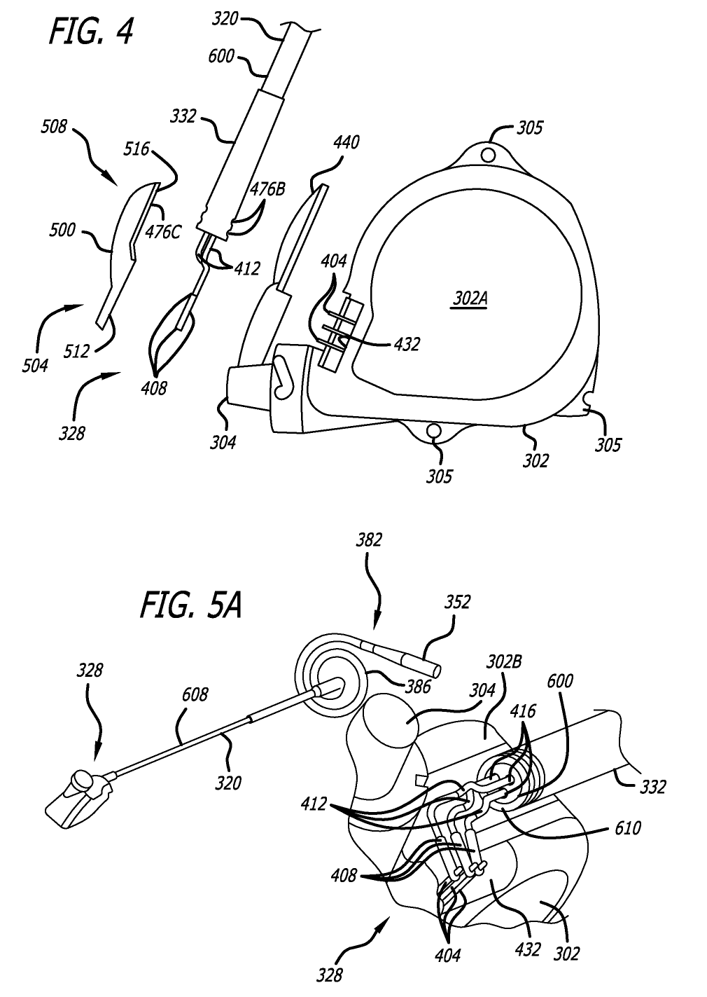

The subcutaneous portion 72 has extending therethrough a plurality of signal lines or wires configured to convey electrical signals to the pump 26 to drive the pump, as discussed further below in connection with FIGS. 4 and 5A.

The communication link 62 can be configured to enhance isolation of at least one of a percutaneous site and the subcutaneous portion 72 from external factors, including the extracorporeal portion 68, which could transfer external forces thereto. As discussed further below, an isolation portion 80 can be disposed between the subcutaneous portion 72 and the extracorporeal portion 68.

As will be discussed in greater detail below, the extracorporeal portion 68, in some embodiments including the isolation portion preferably is configured to be low profile to enable the system to be generally out of the patient's way in use. This provides a benefit of enabling the patient to move around without disrupting the operation of the system 10.

The system 10 also can be configured to manage the transmission of forces along the communication link 62, e.g., transmission of forces to a proximal end of the isolation portion 80. In this context, the “proximal end” is the end farthest from a percutaneous site. Such a configuration preferably prevents transmission of a force or amount of motion that could not be dissipate by the isolation portion 80. For example, in one embodiment, the extracorporeal portion 68 includes a patient lead 100 that extends between the system controller 50 and a proximal end 104 of the extracorporeal portion 68. The patient lead 100 has a first end 108, a second end 112 and an elongate body extending therebetween. The second end 112 is configured to couple with the system controller 50 in any suitable manner to enable control signals from the system controller 50 to be conveyed toward the pump 26.

The first end 108 is configured to couple with the proximal end 104 the extracorporeal portion 68 to be disconnectable under high loads or extreme motion of the extracorporeal portion 18. As discussed further below in connection with FIGS. 10-13, in one embodiment a coupling is provided between the proximal end 104 and the first end 108 that is configured to require a significantly greater force to disconnect the proximal end 104 and the first end 108 than is required to connect these ends. This arrangement enables the break-away load to be selected to be a level below which disruption of at least one of a percutaneous site and a component of the implantable system 14 is likely.

As discussed above, the wearable portion 18 can include the power supply 54 and the data link 58. These components can be made to communicate with the system controller 50 in any suitable fashion, for example by use of electrical leads 120. FIG. 1 shows that the power supply 54 can include one or more of a rechargeable battery 124 or a power adapter 128 coupled with a power source 132. Providing multiple power sources provides redundancy so that the system will not unexpectedly stop operating, e.g., such that the blood-flow through the pump 26 continue undisturbed.

The auxiliary power management system 22 is configured to maintain one or more rechargeable batteries 124 in a charge state such that a patient is able to continue to maintain power to the system controller 50 and the pump 26. The auxiliary power management system 22 can include a power supply 140 and a battery charging cable 144. The battery charging cable 144 can take any suitable form, for example being configured to charge one or more, e.g., two rechargeable batteries 124 at the same time.

B. Application of One Embodiment of an Implantable System

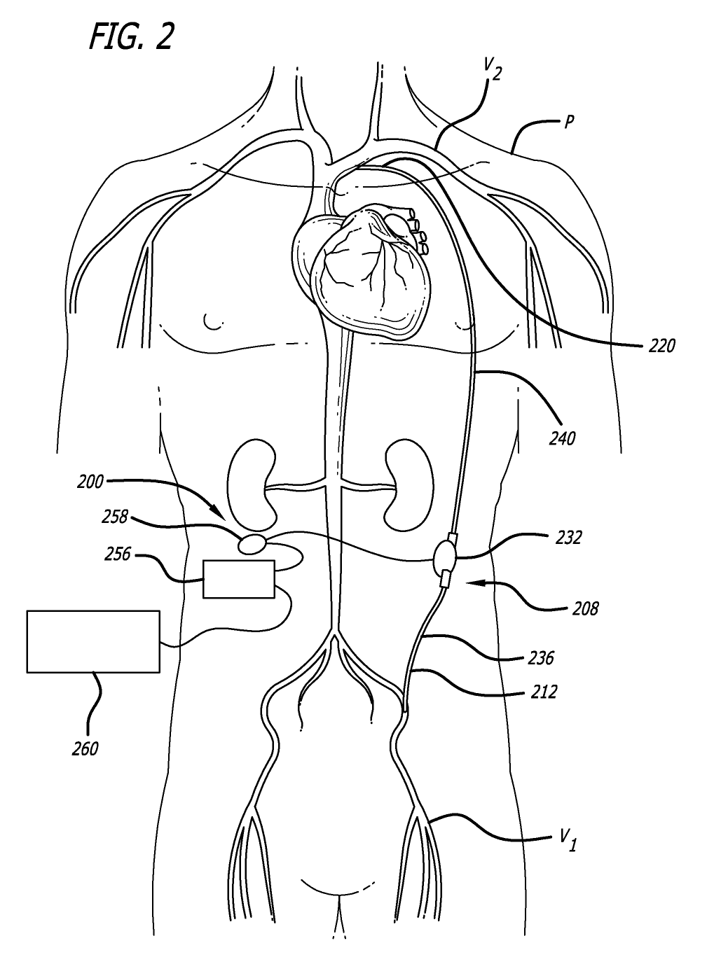

FIG. 2 shows one embodiment of an implantable heart assist system 200 that has been applied to a patient P. The implantable system 200 is configured to convey blood between a first blood vessel V₁ and a second blood vessel V₂ of the vasculature of the patient P. This conveyance of blood can be achieved by a blood flow circuit 208 that extends between the first and second blood vessels V₁, V₂.

In the illustrated application, a first end 212 of blood flow circuit 208 is coupled with an iliac artery while a second end 220 of the blood flow circuit 208 is coupled with an axillary artery. In the embodiment of FIG. 2, the entire blood flow circuit 208 is applied to the patient such that it is implanted beneath the skin of the patient. As used herein, the terms “implantable” and “implantable system” are broad terms that includes systems where all or substantially all of a blood flow conduit are disposed beneath the skin, even if other components of the system are outside of the skin. This term also includes systems that are entirely implanted the beneath the skin, such as where a blood flow conduit and ancillary components thereof, such as controllers and/or power sources, are disposed in the skin.

The blood flow circuit 208 can take many forms. In one embodiment, the blood flow circuit 208 includes an implantable pump 232 that is disposed between the first and second ends 212, 220 of the blood flow circuit. The system 200 can include an inflow blood conduit 236 that is positioned between the implantable pump 232 and the first end 212 as well as an outflow blood conduit 240 that is positioned between the implantable pump 232 and the second end 220 of the blood flow circuit 208.

The blood conduits 236, 240 can take any suitable form, but preferably are made of a biocompatible material such as ePTFE. In one variation of the system 200, the blood conduits 236, 240 are separate components prior to implantation that are assembled during the course of the procedure. In particular, the inflow blood conduit 236 can be configured to be coupled with an inlet port of the pump 232, e.g., using a suitable mechanical connector discussed further below. The inflow blood conduit 236 has an inflow end spaced away from the pump 232 when coupled therewith, which end can be configured to couple with a blood vessel in a suitable manner. For example, as discussed further below a suitable anastomosis, e.g., an end-to-side connection, can be made between the inflow blood conduit 236 and an iliac artery. Also, the outflow blood conduit 240 can be configured to be coupled with an outlet port of the pump 232, e.g., using a suitable mechanical connector discussed further below. The outflow blood conduit 236 has an outlet end spaced away from the pump 232 when applied thereto, which end can be configured to couple with a blood vessel in a suitable manner. For example, as discussed further below a suitable anastomosis, such as an end-to-side connection, can be made between the outflow blood conduit 236 and an iliac artery.

In some variations one or both of the blood conduit 236, 240 are integrated into the implantable pump 232 such that upon application to the patient, the ends of the blood conduit 236, 240 need not be connected to the pump 232 by the clinician.

FIG. 2 shows a suitable approach of the outlet end of the outflow blood conduit 240 to the axillary artery. In particular, in this application, the blood approaches the axillary artery at a relatively small angle with respect to the distal segment of the axillary artery. As used in this context the “distal segment” is the portion of the axillary artery farther from the heart than the point of anastomosis, e.g., toward the patient's arm. This low angle of approach is one that urges blood to flow through the proximal segment of the axillary artery (i.e., the segment between the point of anatomosis and the aorta) toward the aorta. Such a flow direction carries a significant portion of the blood exiting the outflow blood conduit 240 back into the aorta and down the descending aorta. This manner of flow can provide a continuous flow augmentation in the aorta and provide therapeutic benefits associated with such a flow regime, as discussed herein.

The implantable pump 232 can take any suitable form, but preferably is made of biocompatible materials such that it can reside within the patient subcutaneously for the duration of the lifecycle of the system 200.

The system 200 also includes a controller 256, a communication link 258, and a power management system 260. These components can be similar to those hereinbefore described in connection with the system 10.

As discussed in greater detail elsewhere herein, in some embodiments some components of the system 200 are maintained outside of the patient. These components preferably are configured to be relatively small, compact, and a light weight such that the components can be wearable by the patient. For example, in one embodiment, the controller 256 and the power management system 260 are configured to be disposed outside the patient and to be wearable components. The controller 256 preferably provides control signals to the pump 232 by way of the communication link 258. The communication link 258 extends percutaneously between the pump 232 and the controller 256, having a subcutaneous portion and an extracorporeal portion. In some applications, the communication link 258 also is configured to mechanically isolate the pump 232 and other subcutaneous components from components and activity outside the patient.

Having described components of some embodiments of implantable systems for treating cardiac insufficiency and of methods of their application to patients, particular component configurations will now be discussed.

II. Percutaneous Power and Communications Link

As discussed above, the systems 10, 200 provide percutaneous connection between critical components, e.g., between a controller and a pump. A highly reliable connection between these components is desirable, particularly in the ambulatory mode. It is desired to have a highly reliable electrical connection between implanted and extracorporeal components to maintain power and control signals to the implanted components. A highly reliable mechanical connection between implanted and extracorporeal components can ensure that fatigue and other mechanical factors will not compromise the performance of the systems and apparatuses. Such connections enable such components to remain operational unless intentionally taken off-line. Various features that increase the reliability of a percutaneous line are discussed below in connection with various pump assembly embodiments.

A. Pump and Communication Link Assembly

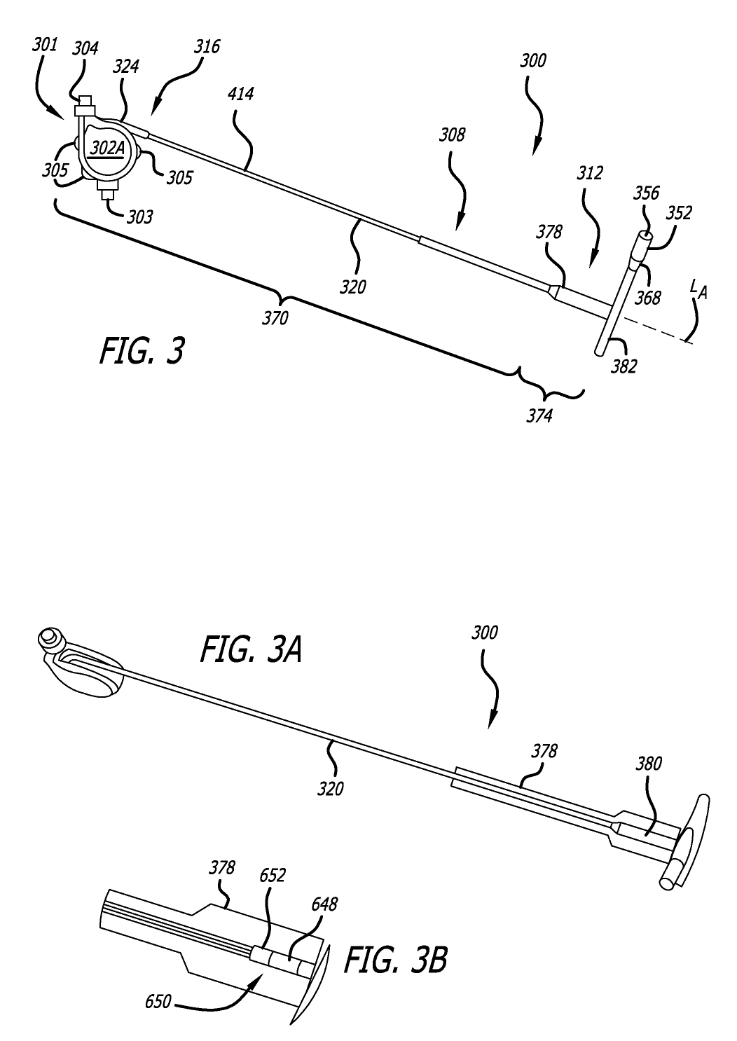

FIG. 3 illustrates a pump assembly 300 that includes an implantable pump 301 and a communication link 308. The pump 301 can take any suitable form, but preferably is adapted for long-term implantation within the patient. For example, the pump 301 can comprise a casing 302 that has a relatively thin configuration for subcutaneous implantation. The casing 302 has a back surface 302A, a front surface 302C, and a relatively thin peripheral side or edge 302B extending from the back to the front surfaces 302A, 302C. The pump 301 also has an inlet port 303 and an outlet port 304 in fluid communication with a pump chamber (not shown). The inlet port 303 conveys blood from an inflow blood conduit into the pump chamber and the outlet port 304 conveys blood from the pump chamber into an outflow blood conduit. The casing 302 and pump surfaces that contact blood preferably comprise titanium or another biocompatible material. The pump 301 preferably also includes one or more securement features 305 disposed about the periphery of the casing 302 for coupling the pump 301 with subcutaneous tissue using suture or another securement device. Further features of the pump 301 are discussed herein below. The pump 301 can also incorporate features discussed in US Publication Number 2005-0084398 and in US Publication Number 2007-0231135, which are incorporated by reference herein and which are included herewith as part of an appendix.

The communication link 308 is configured to convey control signals to the pump 301. The control signals can come from an extra-corporeal controller as discussed above. In some embodiments, the communication link 308 also is configured to convey signals from the pump 301 to outside of the patient for monitoring and analysis of the performance of the pump 301 and associated system. The signals can be conveyed to the data link 58 (see FIG. 1) and to a computer for analysis.

In the illustrated embodiment the communication link 308 has a proximal portion 312, a distal portion 316, and an elongated body 320 extending therebetween. The elongate body 320 has a length selected based on the implantation location of the pump 301 and the location of a percutaneous site through which the communications link extends. In one implant technique, the pump 301 is placed subcutaneously near, but just above the patient's waistline. In another implant technique, a properitoneal pocket is formed adjacent to the iliac artery. One advantageous percutaneous site through which the communication link 308 may pass is the contra-lateral upper quadrant sub-costal region. These locations provide general guidance as to an appropriate length of at least the portion of the elongate body 320 that remains subcutaneous upon implantation. Further discussion of sizing and placement of the communication link 308 is discussed below.

In some embodiments the distal portion 316 includes a distal end 324 that is coupled with the implantable pump 301. As discussed further below in connection with FIG. 4-5A, a header assembly 328 can be provided between the implantable pump 301 and the communication link 308 to provide a robust connection therebetween. The distal portion 316 also can include a grommet 332 or other structure to provide strain relief to maintain the integrity of the communication link 308 and the connection between the communication link 308 and the implantable pump 301. The grommet 332 can be positioned adjacent to the distal end 324 to improve the durability of the distal portion 316 of the communication link 308.

The proximal portion 312 of the communication link 308 can take any suitable form. The proximal portion 312 can include a proximal end 352 that is configured to couple with the patient lead 100 or another signal line. The proximal end 352 can be configured to couple by direct connection with the system controller 50 or another system component. The proximal end 352 can include a socket or recessed portion 356 that is configured to receive a connection portion of a patient lead or other signal conveyance. In the illustrated embodiment, a plurality of connector pins 360 is disposed within the recessed portion 356. As discussed below, the connector pins 360 are also electrically connected to a corresponding plurality of electrical conductors that extend through the elongated body 320. In some embodiments, it is also advantageous to provide a strain relief structure 368 adjacent the recessed portion 356. The strain relief structure 368 can be a grommet or similar structure. Other features and embodiment of the proximal end 352 are discussed below in connection with FIGS. 10-13.

In some embodiments the pump assembly 300 includes an implantable portion 370 and an extracorporeal portion 374. The implantable portion 370 includes the pump 301 and a substantial portion of the length of the elongated body 320. The extracorporeal portion 374 of the pump assembly 300 includes the proximal end 352 and a proximal portion of the elongated body 320.

In one embodiment, the pump assembly 300 also includes a tissue ingrowth structure 378 disposed between end portions of the implantable portion 370 and the extracorporeal portion 374. In the illustrated embodiment, the tissue ingrowth structure 378 overlaps end portions of the implantable portion 370 and the extracorporeal portion 374. The tissue ingrowth structure 378 can take any form that facilitates the acceptance of the subcutaneous portion by the patient's body, particularly adjacent to a percutaneous site. The tissue ingrowth structure 378 preferably promotes sufficient ingrowth of tissue to create a barrier to the ingress of bacteria or other infection-generating agents, or a reaction by the patient's body that results in rejection of an implanted portion of the communication link 308. In one embodiment, the tissue ingrowth structure 378 is configured to extend proximal of the percutaneous exit site when applied. This arrangement prevents tissue from becoming invaginated at the percutaneous site, which can reduce the chance of infection. In some embodiments, the tissue ingrowth structure 378 can be configured to be at the percutaneous site or distal thereof.

FIG. 3A illustrates that in one embodiment the ingrowth structure 378 extends over a strain relief structure 380. FIG. 3B illustrates that the strain relief structure 380 extends over a distal end of a braided structure. These features are discussed in greater detail below.

1. Structure for Isolating Percutaneous Site and Implanted Components

After sufficient tissue ingrowth, it is preferred that the barrier formed thereby be maintained or that disruption of that barrier be minimal. Accordingly, the communications link 308 can be configured to absorb abrupt movements of the proximal end 352 to prevent or minimize disruption of the implantable portion 370 or percutaneous site.

In some embodiments an isolation portion 382 is positioned between the tissue ingrowth structure 378 and the proximal end 352 of the communication link 308. The isolation portion 382 can be configured to absorb or otherwise dissipate movement or loads that may be applied to the proximal end 352. Such movements can occur, for example, when a patient lead is inadvertently pulled on or caught on something or is disconnected from the proximal end 352, either intentionally or unintentionally. In some embodiments, the isolation portion 382 is a generally coiled member that is configured to absorb movement of an amount up to about the length of the coil, e.g., the length of a coiled section when fully uncoiled. In one embodiment, the isolation portion 382 is configured to absorb movement of the proximal end 352 of at least about 1 cm. In another embodiment, the isolation portion 382 is configured to absorb a movement of the proximal end 352 of up to about 10 cm. In another embodiment, the isolation portion 382 is configured to absorb a movement of the proximal end 352 of up to about 20 cm. In another embodiment, the isolation portion 382 is configured to absorb a movement of the proximal end 352 of up between about 20 cm and about 30 cm. In this context, “absorb” is intended to be a broad term that includes both the complete absorption of such movement as well as absorption of substantially all movement while transmitting only a minimal force applied at the proximal end 352.

FIG. 3 illustrates that the isolation portion 382 preferably is a relatively low-profile structure. In one embodiment, the isolation portion 382 is made low-profile by extending primarily in a direction substantially different from that of the implanted portion of the elongated body 320. For example, in one embodiment a length of the elongate body 320 extending distal of the isolation portion 382 extends along a longitudinal axis L_{A} and the isolation portion extends generally transverse to, e.g., substantially within the plane that is generally perpendicular to, the longitudinal axis L_{A}. In FIG. 3, the plane in which the isolation portion 382 extends can be described as being into and out of the page. In another embodiment, the isolation portion 382 is configured to maintain a substantially constant separation along its length from a plane that extends perpendicular to the tissue ingrowth structure 378. The isolation portion 382 can be made generally low-profile by being configured to extend along the patient's skin adjacent to a percutaneous site. Thus, the isolation portion 382 can reside beneath the patient's clothing, out of the way. This further facilitates the preservation of the barrier created by tissue ingrowth at the percutaneous site in the tissue ingrowth structure 378.

The isolation portion 382 preferably also is relatively compact. For example, the isolation portion can be configured to have a length that is greater than a circumference defined by the greatest perpendicular distance from the longitudinal axis L_{A} to any portion of the isolation portion 382. In one embodiment, the isolation portion 382 is made compact by having at least one overlapping portion where a segment of the isolation portion 382 extends adjacent to and along another segment thereof.

Preferably the extracorporeal portion of the elongated body 320 is flexible enough to permit the isolation portion 382 to be lifted away from the skin such that the percutaneous site can be cleaned periodically.

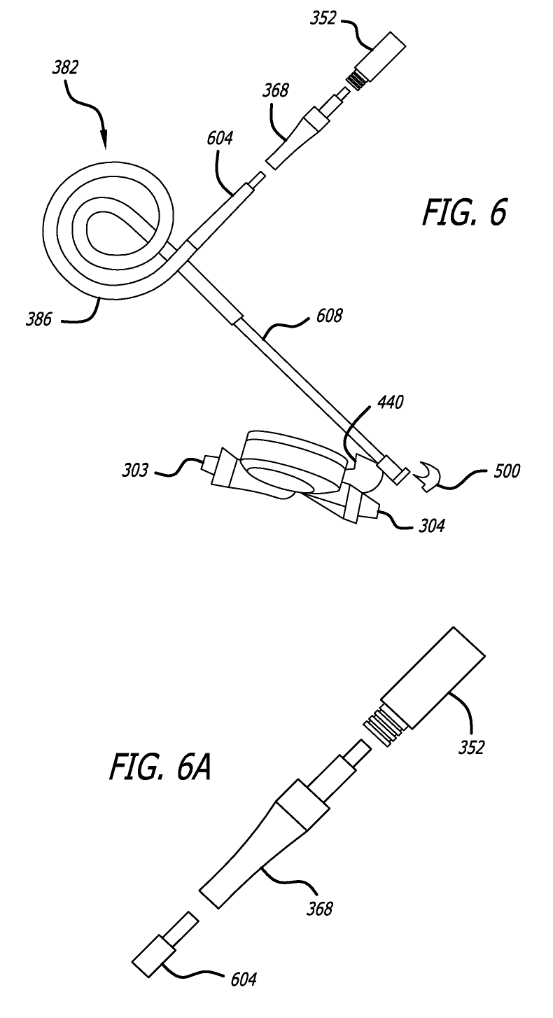

FIGS. 5 and 6 illustrate that one embodiment of the isolation portion 382 includes a proximal length or portion 386 of the communication link 308. The proximal portion 386 has a spiral configuration that at least partially reduces movements of or forces applied to the proximal end 352. Such movements or forces can be attenuated, for example, by being taken up by uncoiling or other displacement of a length of the isolation portion 382. In one embodiment, a spiral portion can become un-coiled due to movement of the proximal end 352 without a subcutaneous portion or the tissue ingrowth structure 378 being subjected to significant forces (e.g., forces that might disrupt the ingrown tissues). In some embodiments, the movements or forces can be attenuated by being stored in a spring-like structure, e.g., through deformation of the structure.

FIGS. 5 and 6 illustrate that the proximal portion 386 can comprise a spiral arrangement subtending an angle of at least about 540°. The proximal portion 386 can be formed into an arcuate structure comprising an angle of greater than 360° in one embodiment. Depending on the properties of the communication link 308 and the degree of movement that should be tolerated by the system, a greater or lesser length of spiraled or coiled conduit can be provided. For example, in one embodiment a spiral argument structure that subtends an angle of at least about 180° would provide sufficient isolation of the tissue ingrowth structure 378 or other percutaneous site interface from forces or movements. In another embodiment a spiral arrangement that subtends an angle of at least about 360° would provide sufficient isolation of the tissue in growth structure 378 or other percutaneous site interface from forces or movements. In another embodiment, a spiral argument that subtends an angle of at least about 720° would provide sufficient isolation of the tissue in growth structure 378 or other percutaneous site interface from forces and movements. Also, other non-spiral configurations could be provided for the isolation portion 382.

The low-profile configuration of the isolation portion 378 and of the proximal portion 312 also help to manage and minimize the effects of forces and movements applied to extracorporeal portions of the pump assembly 300 and of systems associated therewith.

2. Header Structures to Protect Electrical Conductors

FIGS. 4, 5, and 5A illustrate further features of the header assembly 328 discussed above. The header assembly 328 preferably is configured to protect wires that extend within the communications link 308 to minimize the possibility that these signal conveyances may become damaged by movement of the communications link 308 relative to the pump 301.

FIG. 5A illustrates electrical connection between a plurality of contacts 404 associated with the casing 302 and a corresponding plurality of weld crimp pins 408 associated with the communication link 308. The contacts 404 extend from the side edge 302B of the casing 302 and transfer signals between the communications link 308 and internal circuitry of the pump. Each of the weld crimp pins 408 is coupled with a corresponding electrical conductor or wire 412. As discussed further below, in one embodiment, each wire 412 extends within a corresponding lumen 416 defined within the communication link 308 in one embodiment. A robust electrical connection between the communication link 308 and implantable pump 301 protects this electrical connection from the subcutaneous environment, e.g., from potentially fatigue-inducing cycles induced by patient movement, which is transferred to the pump 301 and communications link 308.

In one embodiment the implantable pump 301 comprises a recess 432 that is located on the side edge 302B and that surrounds the base of the contact 404. The recess 432 preferably extends from the area of the contacts 404 along the side edge 302B away from the outlet port 304. The recess 432 is formed to receive therein a base portion 440 of the header assembly 328.

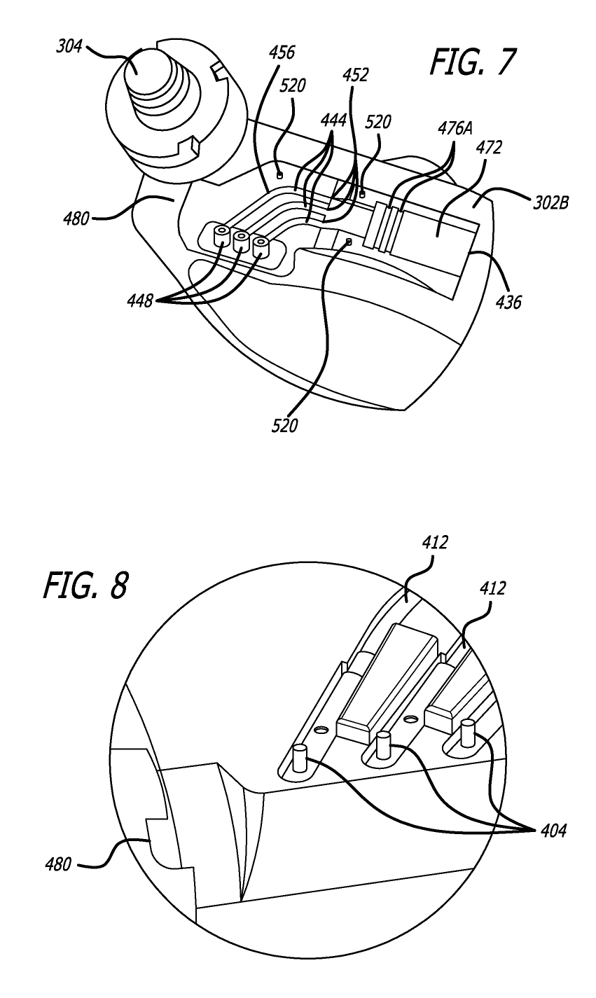

The base portion 440 of the header assembly 328 preferably is molded of a suitable polymeric material and has formed therein a plurality of channels 444 (see FIG. 7) having a first end 448 and a second end 452. The first end 448 is located such that when the base portion 440 is coupled with side edge 302B of the pump 301, the contacts 404 are disposed at the first end. The channels 444 preferably are configured to minimize the likelihood of kinking of the signal wire 412. In one embodiment, the channels 444 comprise a curved portion 456 disposed between the first and second ends 448, 452. The curved portion 456 comprises a radius large enough to prevent kinking or other damage from occurring in the signal wires 412. In one embodiment, the header assembly 328, e.g., the base portion 440, is configured for locating crimp pins 408. In one embodiment, the header assembly 328, e.g., the base portion 440, is configured to prevent loads from being transmitted to the contacts 404. In one embodiment, the header assembly 328, e.g., the base portion 440, is configured to minimize loads that are transmitted to the contacts 404.

The base portion 440 preferably also includes a recess 472 configured to receive a distal portion of the communications link 308. The recess 472 can be configured to receive a distal portion of the grommet 332. In one embodiment the recess includes a plurality of arcuate ridges 476A configured to be received by corresponding argument channels 476B formed in the distal portion of the grommet 332. See FIG. 5B. The engagement of the ridges 476A in the channels 476B minimizes movement of the grommet 332 relative to the side edge 302B of the casing 302.

Engagement of the base portion 440 with the casing 302 can be achieved in any suitable fashion. In one embodiment, the side edge 302B of the casing 302 and the base portion 440 are configured with mating features 480. FIG. 7 shows that a plurality of mating features 480 can be provided in one embodiment. For example FIG. 7 illustrates that four engagement features can be provided. The engagement features 480 preferably are configured to snap together for easy assembly.

In one body, the header assembly 328 also includes a header cover 500 configured to mate with the header base 440. In one embodiment, the header cover 500 includes a distal portion 504 configured to be disposed generally over the contacts 404 and a proximal portion 508 configured to be disposed over at least a portion of the grommet 332. In one embodiment, the distal portion 504 includes a post-connection receiving recess 512 formed in the bottom side of the cover 500. The recess 512 is configured to receive at lease one of the contacts 404. In one embodiment, the recess 512 includes a channel corresponding to each of the contacts 404. When the cover 500 is coupled with the base 440, the recess 512 is dispose over and completely covers the contacts 404. The proximal portion 508 of the cover 500 is configured to mate with the grommet 332, in one embodiment, by having a plurality of protrusions 476C formed within a generally cylindrical recess 516. The recess 516 is configured to receive the body of the grommet 332, which can be cylindrical or cone shaped. The protrusions 476C are configured to mate with the arcuate channels 476B discussed above. The protrusions 476A, 476C meet within the arcuate channels 476B to anchor the grommet 332. By anchoring the grommet 332, the header 328 minimizes or eliminates movement of the communication link 308 relative to the casing 302. By minimizing the movement of the communication link 308 relative to the pump casing 302, fatigue at the signal wire 412 can be minimized or eliminated.

In one embodiment a plurality of posts 520 is formed in the header assembly 328 for coupling the header base 440 and the header cover 500. The posts 520 can be configured to be relatively rigid and to extend upwardly from an upper surface of the base 440. The posts can be configured to mate with corresponding recess is formed, for example, in the bottom surface of the cover 500.

In assembling the pump assembly 300, the base 440 is received within the recess 432 of the casing 302. Connection between the base 440 and casing 302 is achieved by engaging the engagement features 480, discussed above. Thereafter, electrical connection between the contacts 404 and the signal wires 412 is achieved, e.g., by laser welding. Thereafter, the protrusions 476A and the argument channels 476B are engaged to fix the position of the grommet 332 relative to the casing 302. The signal wires 412 are routed through the channels 444 formed in the base 440. Subsequently, the cover 500 is mated with the base 440. Where provided, the posts 520 are aligned with and received within corresponding recesses formed in the cover 500. Also, the protrusions 476C formed in the recess 516 are aligned and mated with the corresponding argument channels 476B.

In some embodiments further securement of the cover 500 to the base 440 is provided. For example additional securement can be provided by positioning an adhesive and/or a solvent between the base 440 in the cover 500. In another embodiment an adhesive gasket can be provided below the base 440, e.g., on the surface of the peripheral edge 302B. In another embodiment the engagement features 480 are configured to interlock with the cover 500 as well as the base 440. This further engagement can be provided by forming undulations in the casing 302, e.g., on this side edge 302B. Similar undulations can be formed on one or both of the base portion 440 and the cover 500 such that it fully engaged the undulations on the casing 302 and on the base 440 and cover 500 provide additional securement.

3. Multilumen Conductor Housings

As discussed above, the communication link 308 provides electrical signals to the implanted pump 301 through a plurality of electrical conductors or wires 412. Two challenges associated with the plurality of wires 412. First, the wires 412 can generate electromagnetic fields that potentially could disrupt the operation of the pump. Electromagnetic fields can produce an antenna effect, whereby the fields potentially reinforce one another and are propagated in all directions toward other components. These fields raise the level of noise in which these other components operate, which can degrade the performance of such other components. Second, the structural integrity of the wires 412 should be maintained to ensure continuous operation of the pump 301.

In one embodiment, the wires 412 comprise a metal-to-metal composite structure, which combines the desired physical and mechanical attributes of two or more materials into a single wire or ribbon system. The composite structure uses an outer sheath structure to impart strength while the core material is designed to provide conductivity to the pump 301. For example, a core comprising silver can be provided that is surrounded by a metallic sheath comprising MP35N® alloy, which is a nonmagnetic, nickel-cobalt-chromium-molybdenum alloy. In other embodiments, different materials can be used to provide an electrically conducting core and a strength enhancing sheath. Composite wire structures are available through Fort Wayne Metals and are marketed under the trademark DFT® Wire.

In one embodiment the signal wires 412 are disposed within a protective sleeve 414 that comprises a distal end 600, a proximal end 604, and an elongated body 608 extending therebetween. The sleeve 414 can take any suitable form, preferably being configured to withstand anticipated forces, stresses, and duty cycles to be applied to the communications link 308 due to movements of the ambulatory patient.

In one embodiment, the sleeve 414 is configured with multiple lumens 416, discussed above, through which the signal wires 412 extend. The signal wires 412 can be disposed in one or more lumens 416 of the sleeve 414. In one embodiment, the sleeve 414 includes a separate lumen 416 for each signal wire 412. In the illustrated embodiment, the sleeve 414 comprises three separate lumens, one for each of the signal wires 412.

Arrangement of the lumens within the sleeve 414 can take any suitable form. For example, one or more of the lumens 416 can comprise a helical arrangement. The helical arrangement provides a first benefit of cancelling or substantially reducing the strength of an electromagnetic field generated by the signal wires 412. In one embodiment, each of the lumens 416 comprises a helical arrangement whereby each of the lumens comprises a helical arrangement. In one embodiment, one or more lumens 416 comprises an at least partially helical arrangement. In a partially helical arrangement, a portion of the length of the lumen(s) 416 is helical, e.g., adjacent to the pump 301. In a partially helical arrangement, a portion of the length of the lumen(s) 416 is non-helical, e.g., providing a straight length. A relatively short straight length can be provided adjacent to one or both of the proximal and distal ends 600, 604. A longer straight length can be provided away from the pump 301 or other components that could be disrupted by electromagnetic fields.

In one embodiment, the cancellation or reduction of such fields is a primary factor in the design of the arrangement of the lumens 416. Electromagnetic field cancellation can be achieved by providing a helical arrangement of approximately two to four turns per foot. In another embodiment, the helical arrangement is at least about two turns per foot. In another embodiment, the helical arrangement is not more than about four turns per foot. These arrangements provide significant noise reduction or cancellation benefits that enable the components of the systems 10, 200. Reduction of noise enhances the performance of components of the system, e.g., by enabling the system to operate at lower signal levels and at lower power draw from the related power supplies.

The spiral arrangement enhances the strength of the sleeve 414 and the amount of protection provided by the sleeve to the signal wires 412. The signal wires 412 can either be embedded in the lumens 416. In one embodiment, the signal wires 412 can extend through the lumens 416 in a manner that permits the wires to slide relative to the lumens 416. The ability to slide in this fashion results in reduced compressive and tensile forces being applied to the wires 412 as the sleeve 414 ilexes. This reduces the stresses applied to the wires 412 and thereby improves the reliability of the communication link 308.

In one embodiment, the sleeve comprises a visible indicator 610 that assists in assembling the pump assembly 300. For example, a black stripe can be provided along one of the lumens 416 to indicate the location of the wire in that lumen. This enables the assembler to know that which signal wire 412 is connected to which contact 404 of the pump 302. This is particularly useful because the pump 301 is configured to function by rotating in a pre-determined direction Proper connection of the signal wires 412 to the contacts 404 ensures that the pump 301 operates in the proper direction. Because the sleeve 414 is configured with a distinct visual appearance, the location of the lumen indicated can be easily verified.

In one embodiment, the sleeve 414 is shaped by an outer structure 624 that can be a polymeric overmold in one embodiment. The shaping of the sleeve 414 along at least a portion of it's length is a way to provide an isolation portion, e.g., by inducing a selected shape such as a spiral shape or other low profile and compact arrangement, as discussed above. The overmold also reinforces the sleeve 414 to further protect the signal wires 412. provided with reinforcement to further strengthen the communication link 308.

The outer structure 624 can be overmolded with silicone or another suitable biocompatible material. Preferably the outer structure 624 completely encapsulates at least a portion of the length of the sleeve 414 and a reinforcement structure 648 that can be disposed between the sleeve 414 and the outer structure 624. Full encapsulation of the reinforcement structure 648 and/or the sleeve 414 can be provided by positioning a plurality of spacers 616 along at least a portion of the length of the sleeve 414. In one embodiment, seventeen spacers 616 are provided along a length of the sleeve 414 at regular intervals, e.g., about every 0.5 inches. The spacers 616 can be coupled to the sleeve 414 in any suitable manner. In one embodiment, spacers 616 are coupled with the sleeve with a suitable adhesive, such as NUSIL MED-1511.

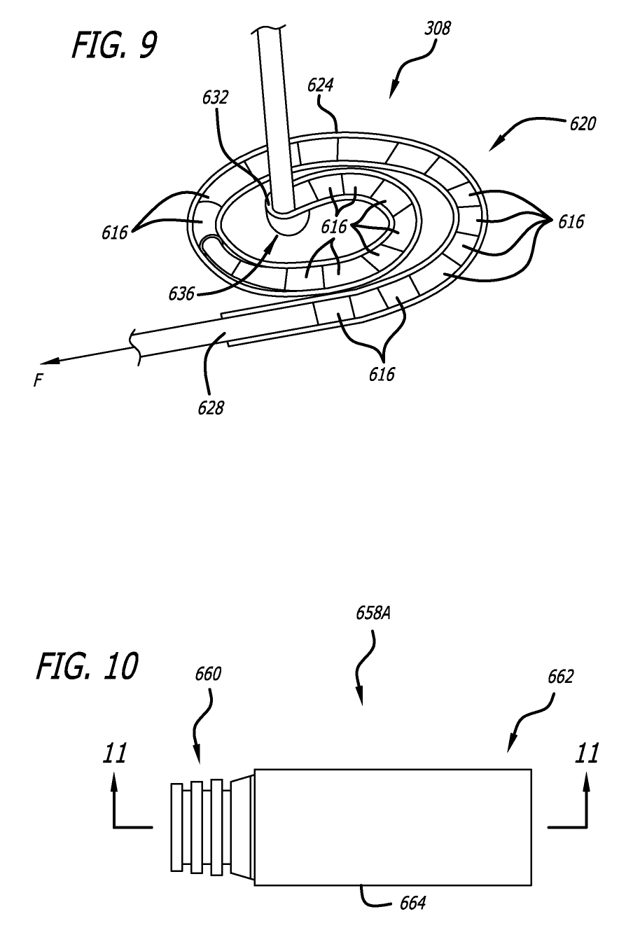

The location of the spacers 616 can vary. In one embodiment the communications link 308 includes a spiral assembly 620. The spiral assembly 620 is one embodiment of an isolation portion, discussed above. The spiral assembly 620 includes a spiral arranged or coiled length of the communication link 308. In the embodiment of FIG. 9, the plurality of spacers 616 is positioned along the coiled length. In one embodiment the spacers 616 are spaced at regular intervals along the coiled length. The spacers 616 are used to space the sleeve 414 and the reinforcement structure 648 from inner walls of a mold in which the outer structure 624 is formed if this structure is overmolded. By providing separation between the walls of the mold and one or both of these portions (if the reinforcement structure is provided), the underlying structures will necessarily be encapsulated in the overmolded outer structure 624.

The outer structure 624 can extend along any suitable length of the coiled portion, for example along the entire length of the coiled portion. In one embodiment the outer structure 624 comprises a proximal end 628 that is located proximal of the coiled portion and a distal end 632 that is located adjacent the distal end of the coiled portion. In one embodiment the distal end 632 of the outer structure 624 is located distal of a transition zone 636 located between the coiled portion and a distal portion of the communications link 308. The transitioned portion 636 transitions the direction in which the signal wires 412 extend from generally the plane of the coiled portion to generally along the longitudinal axis L_{A} of the distal portion of the communications link 308. The transitioned portion 636 facilitates a low profile arrangement of the spiral assembly 620 when the communications link 308 is applied to the patient.

Further reinforcement can be provided by disposing the sleeve 414 within a reinforcement structure 648. For example, the reinforcement structure 648 can include a cylindrical braided structure that extends along at least a portion of the length of the communications link 308. In one embodiment the reinforcement structure 648 extends at least along the length of the spiral assembly 620. Within the spiral assembly 620, the reinforcement structure 648 can be disposed between the spacers 616 and the sleeve 414. In the spiral assembly 620, the reinforcement structure 648 can absorb at least a portion of a force or a movement of a proximal end of the communications link and therefore prevent such force or movement from being transferred to the sleeve 414 and/or to the signal wires 412. One embodiment, the reinforcement structure 648 extends at least to the transitioned portion 636 to protect at lease one the sleeve 414 and the signal wires 412 as these structures transition from the plane of the spiral assembly 620 to along the direction of the longitudinal axis L_{A}.

FIG. 3B shows that in one embodiment the reinforcement structure 648 terminates adjacent to where the percutaneous site is located when the communication link 308 is applied to the patient. In particular, the reinforcement structure 648 has a distal end 650 that is located between the isolation portion 382 (e.g., between the spiral assembly 620) and the distal end of the tissue ingrowth structure 378. Preferably the distal end 650 of the reinforcement structure 648 is encapsulated in a shield member 652 to minimize the chance of a sharp portion of the reinforcement structure 648 being exposed to the patient's tissue. The distal end 650 and the shield member 652 can be disposed beneath the strain relief structure 380 in one embodiment, as illustrated by FIG. 3A. In another embodiment, the reinforcement structure 648 extends at least along a distal portion of the communications link 308, e.g., including at least a portion of a subcutaneous portion of the communications link 308.

As discussed above, the spiral assembly 620 provides the advantage of being able to absorb at least a portion, e.g., a substantial portion or substantially all, of the movement of or force applied to the proximal end 352 of the communication link 308. FIG. 9 illustrates such a force F applied proximal of the spiral assembly 620. The spiral assembly responds to the force F by straightening out, e.g., by un-coiling. As a result the arcuate length-of the spiral assembly 620 becomes generally straightened, permitting the distance between a percutaneous exit site and the proximal end 352 of the communication link 308 while exerting relatively little force at the skin exit site. The spiral assembly 620 preferably has shape memory such after the force F is removed, the spiral assembly tends to return to a coiled shape. In other embodiments, fewer or more turns can be provided as discussed above. Also, in some embodiments of the isolation portion 382 absorption of forces or movement is achieved without a coiled or spiral portion. In such other embodiments, there can also be a shape memory such that upon release of a force F the isolation portion 382 releases stored movement or force to return to a pre-determined shape or configuration.

B. Communication Link and System Controller Coupling

The systems 10, 200 discussed above can also be configured to maintain robust connection between electrical components, such as between a system controller and a communication link. For example, as discussed below, one embodiment of a electrical connection between the communication link 308 and the patient lead 100 provides a convenient keyed coupling and also provides a connection that is easy to connect and that resists inadvertent disconnection.

1. Keyed Connector

In one embodiment, the proximal end 352 is configured to mate with a corresponding connector portion coupled with a system controller. For example, as discussed above, the patient lead 100 can be disposed between the system controller and a communication link. In one embodiment, the proximal end 352 and the first (or distal) end 108 of the patient lead 100 can be configured as mating connectors. In one embodiment, the proximal end 352 and the first end 108 can be configured to be “keyed” in the sense that they are configured to only mate in particular orientation. In one embodiment, the proximal end 352 and the first end 108 of the patient lead 100 are configured to mate in as many configurations as there are signal wires 412 disposed provided in the communications link 308. In the illustrated embodiment, there are three signal wires 412 and three orientations in which the proximal end 352 and the patient lead 100 can mate.

FIGS. 10-13 illustrate a multi-lobular connector allowing for connection in any of three relative orientations of proximal and distal connector portions 658A, 658B. The proximal connector portion 658B can be associated with an external component, such as the patient lead 100. In one embodiment the proximal connector portion 658B forms a portion of the first end 108 of the patient lead 100. The distal connector portion 658A can comprise the portion of the proximal end 352 of the communications like 308. The proximal and distal connector portions 658A, 658B can be configured such that the connection therebetween can be achieved when proximal and distal connector portions are axially aligned into any one of three positions, as discussed further below.

FIG. 10 illustrates that the distal connector portion 658A can comprise a distal portion 660, a proximal portion 662, and an elongate body 664 extending therebetween. The distal portion 660 can be coupled with a proximal portion of the communications link 308. For example, the distal portions 660 can include a plurality of, e.g., three, recesses that are configured to mate with corresponding arcuate ridges (not shown) on an inside surface of the strain relief structure 368. The proximal portion 662 defines an opening providing access to a recessed portion 666. The recessed portion 666 is configured to receive a protruding portion 659 of the proximal connector portion 658B.

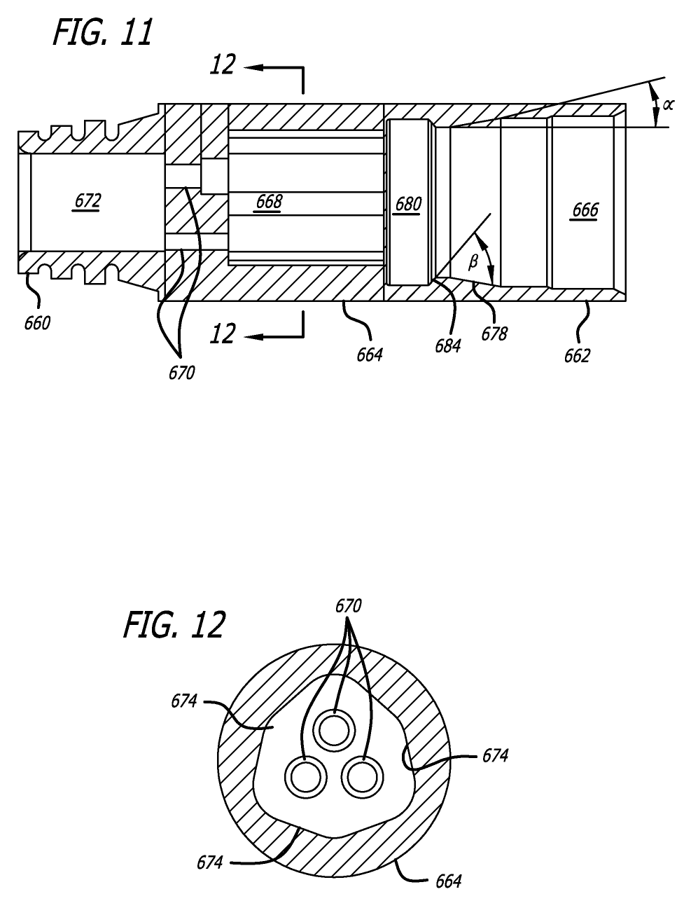

The recessed portion 666 includes a distal end 668 in which a plurality of contacts can be disposed. The contacts are not shown in FIG. 11, but are similar to those shown in FIG. 6A, discussed above. In one embodiment a plurality of channels 670 is formed distal of the distal end 668 of the recessed portion 666. The channels 670 permit contacts to extend proximally through a distal recess 672 into the distal end 668 of the recessed portion 666.

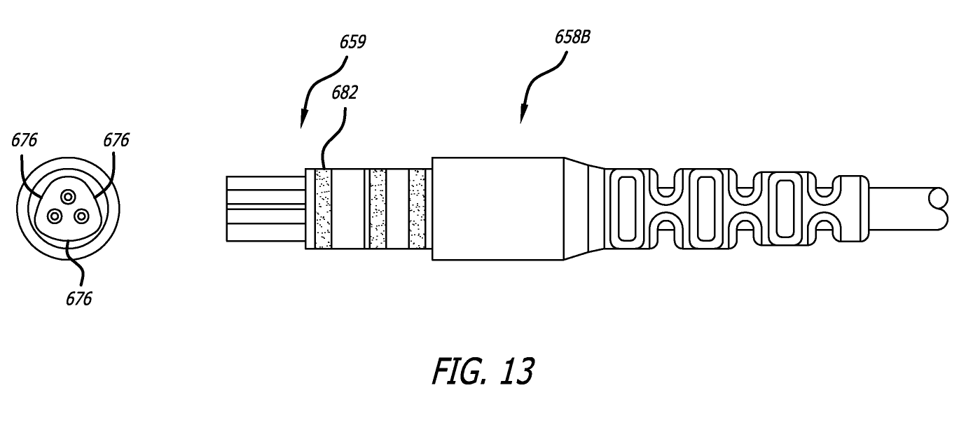

FIG. 12 illustrates that in one embodiment the distal connector portion 658A includes a plurality of surfaces 674 that promote axial alignment between the distal connector portion 658A and the proximal connector portion 6588 to enable these portions to be coupled together while protecting the contacts in the distal end 668. FIG. 13 illustrates that the proximal connector portion 658B has a plurality of surfaces 676 that can have a similar shape to that of the surfaces 674. Axial alignment of the proximal connector portion 658B with the distal connector portion 658A occurs when the surfaces 676 are aligned with the internal surfaces 674. When so aligned, the protruding portion 659 can be advanced into the distal end 668 the recessed 666.

Each of the surfaces 674 is identical, and each of the surfaces 676 is identical, accordingly any of three axially oriented positions can enable the proximal and distal connector portions 658A, 658B to be coupled together. By providing multiple connection orientations a user can more quickly couple the proximal and distal connector portion 658A, 658B. This convenient arrangement enables the connector portions to be assembled quickly, to make a procedure go more quickly and also enable a patient to reconnect disconnected connector portions quickly.

One advantage of making the proximal and distal connector portions 658A, 658B is that the contacts portion can be aligned so that there is no chance of the contacts positioned in the distal end 668 of the recess not being properly coupled with contact on the protruding portion 659 of the proximal connector portion 658B. This can prevent a user from damaging the contacts when connecting the proximal industrial connector portion 658A, 658B.

2. Configured to be Coupled/Decoupled with Relatively Little Insertion Force and Relatively Higher Removal Force

In one embodiment, the protruding portion 659 of the proximal connector portion 658B and the recessed portion 666 of the distal connector portion 658A are configured to be connected with a lesser force and is required to disconnect the distal and proximal connector portions 658A, 658B. In one embodiment, at least about twice as much force is needed to disconnect the distal and proximal connection portions 658A, 658B as is needed to connect these components. In one embodiment, about 2.5 times as much force is needed to disconnect the distal and proximal connection portions 658A, 658B as is needed to connect these components.

FIGS. 11 and 13 illustrate one technique for providing a connection that requires less insertion force then the force required to disconnect the connection. In particular, the elongate body 664 defines a first ramped surface 678 formed in the recessed portion 666. The first ramped surface 678 is located between the opening to the recessed portion 666 and the distal end 668 of the recessed portion. The ramped surface 678 preferably includes a relatively shallow angle surface. For example, the ramped surface 678 can't form an angle between just greater than 0° to 20° in one embodiment. The angle α is measured with respect to a line parallel to the longitudinal axis of the recess 666. Because the ramped surface 678 is relatively shallow, the insertion force when the proximal connection portion 6588 is advanced into the connection portion 658A is relatively small.

Once the distal end of the protruding portion 659 of the proximal connection portion 658B is advanced past the ramped surface 678 the distal end of the protruding portion 659 is advanced into and resides within a connection zone 680. The connection zone 680 is located between the ramped surface 678 and the distal end 668 of the recessed portion 666.

In one embodiment, the distal end of the protruding portion 659 includes expandable member 682. The expandable member 682 can take any suitable form and in one embodiment is a helical spring. The expandable member 682 is compressed upon the bringing of the distal end of the protruding portion 659 into engagement with the ramped surface 678. As the protruding portion 659 is advanced toward the distal end of the ramped surface 678, the expandable member 682 becomes progressively more compressed. After the distal end of the protruding portion 659 reaches the connection zone 680, the expandable member 682 expands outwardly toward its uncompressed state.

In one embodiment the distal connection portion 658A includes a second ramped surface 684 that is located just proximally of the connection zone 680. The second ramped surface 684 is relatively steep compared to the first ramped surface 678. In one embodiment, the second ramped surface 684 forms an angle β with respect to the longitudinal axis of the recessed portion 666 that is greater than the angle α. In particular, the angle α is approximately 10° and the angle β between approximately 60°. In another embodiment the angle α is approximately 10° and the angle β is approximately 60°. In another embodiment, the angle beta can range from 45 to 75°.

By providing a relatively low insertion force the percutaneous conduit 100 can be relatively easily connected to the communication link 308. This arrangement enables a user to quickly and easily connect the components of the system 200 or of the system 10. Because a much greater force is needed to disconnect the distal and proximal connection portions 658A, 658B a protection against inadvertent disconnection is provided. This is greatly advantageous in that it is preferred that the pump being driven by signals conveyed through the connection portions 658A, 658B not unintentionally cease its operation. While such an event would not be life threatening, if disconnected for lengthy periods the pump or the system may become inoperative.

III. Methods of Implantation

FIG. 2 illustrates, as discussed above, one application of the system 200 to a patient. FIG. 14 illustrates further details of methods for implanting the system 200 and related systems. Prior to any phase of a method specific to the systems discussed herein, standard steps should be taken to prepare the sterile field and the patient for surgery.

Thereafter, in one technique for implanting the system 200, a subcutaneous space 690 is created into which an implantable pump can be placed. The subcutaneous space 690 may be formed in any suitable manner. For example an incision 692 may be made in the skin to access a subcutaneous area. The subcutaneous space 690 may be created by separating adjacent layers of tissue just beneath the skin to form the space therebetween. In one technique a deeper space can be formed, for example, adjacent the peritoneum. In one embodiment, the peritoneum is not penetrated and the pump is placed adjacent to the iliac artery. This technique has the advantage of locating the pump close to the iliac artery such that an inflow conduit can be connected to the iliac artery without the need for tunneling the inflow conduit.

In a subsequent phase of a method, a pump such as the pump 301 can be placed beneath the skin within the subcutaneous space 690. The location of the space 690 and the orientation of the pump 301 when placed therein can be selected to maximize patient comfort. Relevant factors include body habitus, angle between costal margins, clothing lines (e.g., waist bands), and changing body positions (e.g., bending and sitting upright).

In one technique, the communications link 308, is oriented such that an external portion thereof is directed superiorly from its exit site in the mid-clavicular line, 4-6 cm below the costal margin (near-vertical orientation) for males. For females, the percutaneous conduit is to be directed more laterally, about 30° off-vertical to avoid interference with the breast. To prevent the percutaneous conduit from rubbing against the costal margin the distance between the percutaneous exit site and the costal margin should be adjusted based on the thickness of subcutaneous tissue.

Thereafter, a percutaneous conduit exit site or percutaneous site 694 is created by excising a skin button that is approximately half the diameter of the communications link 308. After the percutaneous site 694 has been excised, the communications link 308 can be tunneled from the subcutaneous space 690 to the contralateral upper quadrant of costal region to provide the desired positioning and orientation.

In one technique the communication link 308 is passed through a pathway or tunnel 698 that is formed between the subcutaneous space 690 and the percutaneous site 694. The tunnel 698 can be curvilinear in one embodiment. In one technique, the tunnel 698 is just superior or inferior to the umbilicus, depending on the patient's anatomy. Preferably, the percutaneous tunnel 698 maximizes the length of the path through the abdominal wall muscle (e.g., a path at least 10-12 cm long), entering the muscle within 4-8 cm from the pump, exiting the muscle through a cruciate incision in the fascia, immediately deep to the percutaneous site 694.

A. Tunneling Percutaneous Conduits

Certain embodiments of percutaneous conduits that can be used in the systems described herein make tunneling from the subcutaneous space 690 challenging. For example, the tissue tunnel is to be maintained relatively narrow, whereas certain embodiments of the communication link 308 (e.g., having a spiral portion) have a much wider profiles. A tissue tunnel approximately equal to the transverse size of the spiral of the isolation portion 382 would not be practical. Also, the isolation portion 382 of the communication link 308 is relatively flexible, as discussed above, which would make urging the communications link 308 through a subcutaneous tunnel difficult. Also, subsequent connectability of the proximal portion 352 to a patient lead could be complicated by directly contacting bodily fluids or tissue in and around the tunnel.

Accordingly it would be useful to provide a device for enabling percutaneous components, such as the communication link 308, to be drawn through tissue beneath the skin. In some techniques, such a device can be configured to be pulled through a pre-formed tunnel, as discussed below.

1. Percutaneous Conduit Tunneling Apparatus

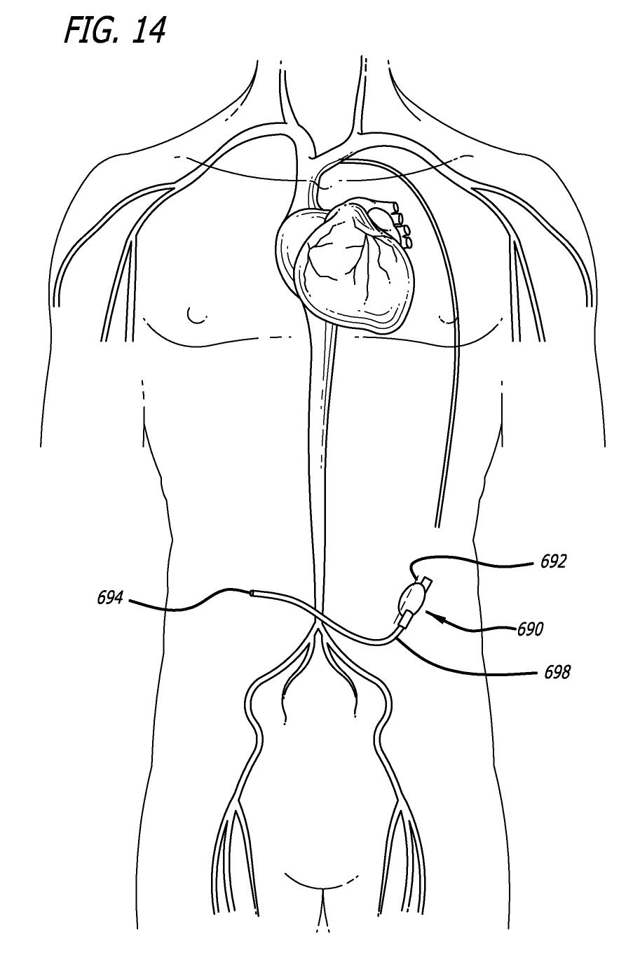

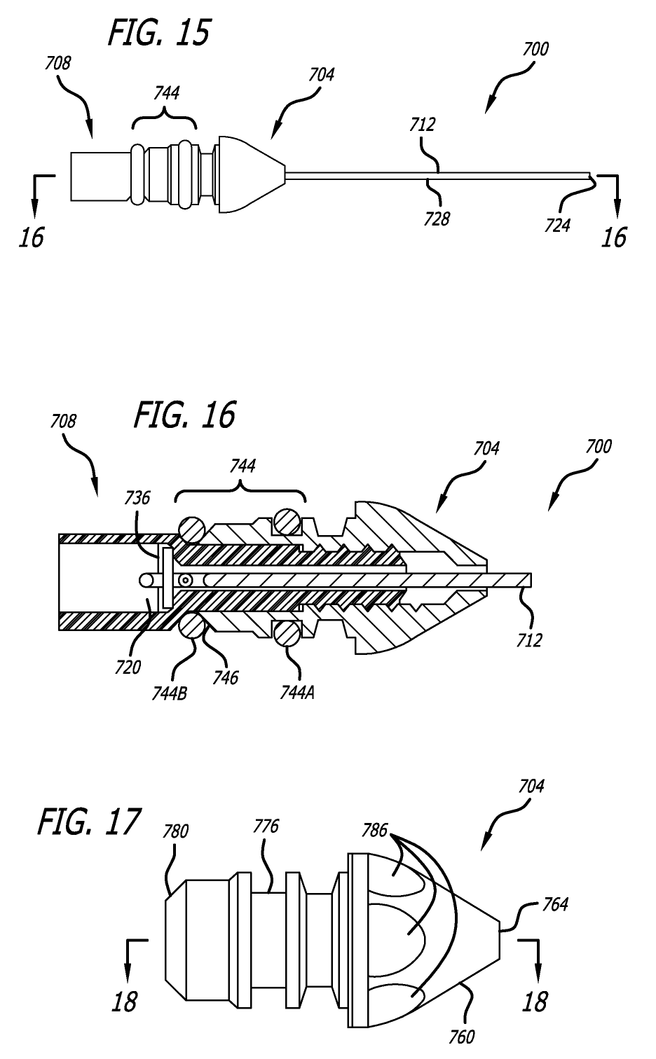

FIG. 15 shows one embodiment of the tunneling apparatus 700 that can be used to convey the proximal end of a percutaneous conduit, such as the communications link 308, through a tissue tunnel from adjacent to the subcutaneous space 690 to the percutaneous site 694. In one embodiment the tunneling apparatus 700 includes a leading portion 704, a trailing portion 708, and a tension member 712.

The tension member 712 can take any suitable form, but preferably includes a first end 720, a second end 724, and an elongate portion 728 that extends between the first and second ends 720, 724. In one embodiment, the proximal end 724 is anchored to at least one of the leading portion 704 and the trailing portion 708. For example, an anchor 736 can engage the first end 720 of the tension member 712 to retain the tension member within the trailing portion 708, as shown in FIG. 15. Further details of the anchor 736 are discussed below in connection with FIG. 15.

FIGS. 15 and 16 illustrate that the tunneling apparatus 700 is configured to isolate the proximal end of a percutaneous conduit from body fluids and tissues to which it would be exposed when pulled through the pertaining is tunnel. In one embodiment, the proximal end of the percutaneous conduit is isolated from such tissues and fluids by a seal structure 744 of the tunneling apparatus 700. The seal structure 744 can take any suitable form but preferably is configured to prevent the ingress of tissues and fluids into at least one of internal portions of the tunneling apparatus 700 and proximal portion of the percutaneous conduit to which the tunneling apparatus is coupled.

In one embodiment seal 744 includes a first seal member 744A and a second seal member 744B. The first seal member 744A can be disposed forward of the second seal member 744B. In one embodiment the first seal member 744A can be coupled with the leading portion 704 and can be configured to provide a seal with an inner portion of the proximal portion of the pertaining is conduit with which the tunneling apparatus 700 is coupled. For example, the first seal member 744A can comprise an O-ring that is seated on the leading portion 704 and that is dimensioned to form a sealing engagement with a proximal portion of the percutaneous conduit, e.g. with a recess or socket in the proximal end 352 of the communication link 308. For example, in one technique, the trailing portion 708 is advanced into the proximal end socket of the communication link 308 until the proximal end 352 is forward of the first seal member 744A. In this position, a seal can be formed between the tunneling apparatus 700 and the proximal end socket of the communications link 308.

The second seal member 744B can provide a further sealing function that can be distinct from or supplemental to the sealing function of the first seal member 744A. For example, in one embodiment the leading portion 704 and the trailing portion 708 are members that can be separated from one another. Interconnectability can result in one or more gaps 746 forming between components of the leading portion 704 and the trailing portion 708. The gap 746 could permit bodily fluids or tissues to enter internal spaces of the tunneling apparatus 700. In one embodiment the seal member 744B is an O-ring that is disposed at the gap 746 to prevent the ingress of fluids or tissues during the course of tunneling.

In one embodiment the seal member 744B can also be configured to engage with an internal surface of a proximal end socket of a percutaneous conduit, such as the communications link 308 to provide enhanced engagement between the tunneling apparatus 700 and the percutaneous conduit to which it is coupled. In some applications and methods, the force needed to pull the tunneling assembly 700 through a tissue tunnel can be relatively high. Accordingly, it is desirable to enable the tunneling assembly 700 to engage the percutaneous conduit sufficiently strongly such that the tunneling assembly does not become disconnected from the percutaneous conduit in use. One way to provide a relatively high grip between the tunneling assembly 700 and a percutaneous conduit is to configure the seal member 744B to expand into engagement with an inner surface of a recess formed at the proximal end of the conduit. Such expansion can create a frictional engagement that will not be overcome by the forces encountered in pulling the tunneling apparatus 700 and conduit with which it is coupled through a tissue tunnel. Expansion of the seal member 744B can be achieved in any suitable manner, such as by axially compressing the member to create radial expansion. This approach is discussed further below.

In one embodiment the primary function of the seal member 744B is to provide a secure engagement with a percutaneous conduit and a secondary function is to provide redundancy in the seal between the outer surfaces of the tunneling apparatus 700 and an inner surface of a percutaneous conduit. Preferably the seal member 744B is dimensioned to mate with the internal surface of the percutaneous conduit in a manner that would prevent fluids or tissues from moving past the seal member 744B.

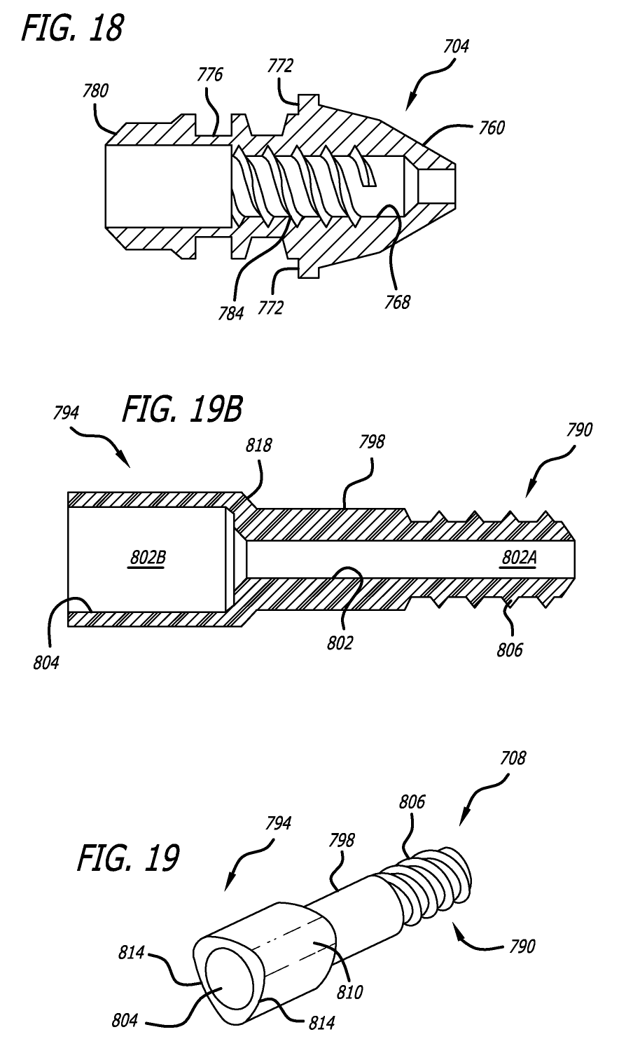

FIGS. 17 and 18 show further details of the leading portion 704 of the tunneling apparatus 700. The leading portion 704 includes an angled surface 760 that extends rearwardly from a forward end 764 and a lumen 768 formed through the leading portion. The angled surface 760 can be configured to move tissue that is located in front of the leading portion 704 latterly away from the tunneling apparatus 700 so that the tunneling apparatus can be drawn through the tissue from the subcutaneous cavity 690 toward the percutaneous site 694. In one arrangement, the angled surface 760 is at least partially conical. In one embodiment, the angled surface 760 is formed at approximately a 30° angle to a central longitudinal axis of the lumen 768.

A rear facing surface 772 is provided on the leading portion 704 at a location rearward of the angled surface 760. Further rearward of the rear facing surface 772, the leading portion 704 includes a recessed portion 776 and a forwardly angled seal engagement surface 780. As discussed further below, the rear facing surface 772 is configured to provide an abutment up against which a proximal end of the percutaneous conduit can be advanced such that the clinician can confirm engagement between the tunneling apparatus 700 and the conduit. The seal engagement surface 780 is angled such that a rearward portion of that surface can be received beneath a portion of the seal member 744B, as shown in FIG. 16.

The recessed portion 776 can take any suitable form, but preferably is configured to receive the seal member 744A therein. In one embodiment the depth of the recessed portion 776 is selected to be less than the height of the seal member 744A. In this arrangement, a least a portion of the seal member 744A extends beyond the structure defined the recessed portion 776 to a position where it can engage an internal portion of a proximal portion of a percutaneous conduit. In one embodiment, at least one of the forwardly angled surface 780 and the recessed portion 776 comprises a corresponding sealing surface to enhance a fluid and or tissue tight seal.

FIG. 18 shows that in one embodiment the lumen 768 also includes internal threads 784 that are configured to provide secure engagement between the leading portion 704 and the trailing portion 708 of the tunneling apparatus 700. The internal threads 784 can take any suitable form, for example comprising double start threads with a suitable pitch. In one embodiment double start threads with a 16′pitch is provided.

FIG. 17 shows that in one embodiment a plurality of tooling flats 786 can be provided on the tunneling apparatus 700 to enable the leading and trailing portions 704, 708 to be decoupled from each other or from a percutaneous conduit which to which it is coupled. As discussed above, threaded engagement between the leading and trailing portions 704, 708 is one way to enhance securement between the tunneling apparatus 700 and a conduit to be tunneled. The tooling flats can be provided on the leading portion 704 to enable a torque generating tool to be coupled with the leading portion so that a clinician can more easily decouple the leading and trailing portions 704, 708 from each other. In one technique the leading portion is disengaged from the seal member 744B such that the seal member 744B becomes un-stressed axially permitting the seal member 744B to assume a configuration having a smaller radial size.

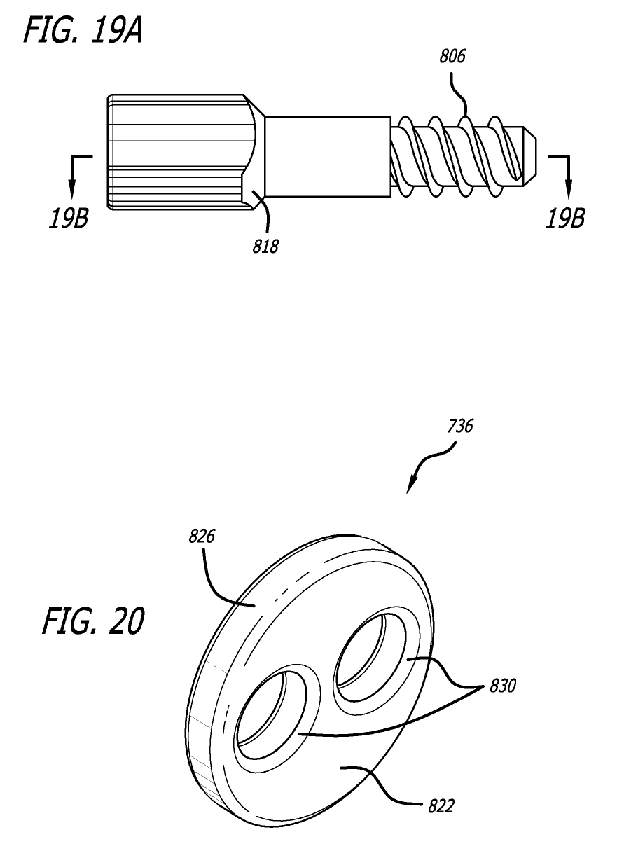

FIGS. 19-19B show further details of the trailing portion 708. For example, the trailing portion 708 including first end 790, a second end 794, and an elongate body 798 that extends therebetween. A lumen 802 extends through the elongate body 798 between the first and second ends 790, 794 and includes a forward portion 802A a rearward portion 802B. The forward portion 802A can take any suitable configuration, and in one embodiment is approximately the size of the tension member 712. The rearward portion 802B of the lumen 802 can take any suitable form but preferably defines a recess 804 that is large enough to receive delicate structures located in the proximal portion of a percutaneous conduit. For example, the communications link 308 includes a plurality of contacts, which can be received within the recess 804 when the conduit is coupled with the tunneling apparatus 700.

The first end 790 preferably includes threads 806 configured to meet with the thread 784. The second end 794 preferably includes an enlarged body 810 that is configured to mate with a proximal portion of the communication link 308 or another percutaneous conduit. Similar to the arrangement of the proximal and distal connector portions 658A, 658B, the communications link 308 and the enlarged body 810 can be configured with a keyed arrangement whereby rotational alignment of the enlarged body 810 and the communication link 308 precedes engagement. In one embodiment, the trailing portion 708 and a proximal portion of the percutaneous conduit can be configured to be coupled in any of the plurality of radially aligned positions. For example, one embodiment provides a tri-lobular construction in which the enlarged body 810 comprises three lobe surfaces 814 disposed about the body 810. The lobe surfaces 814 are configured to mate with lobe-like surfaces defined within the proximal portion of a percutaneous conduit, as discussed above.

In one embodiment, the trailing portion 708 and the leading portion 704 are separate components that can be coupled together. For example, as discussed above the trailing portion can be connected to the leading portion 704 by engaging the threads 806, 784. In this arrangement, a seal member 744B can be provided between the leading portion 704 and the trailing portion 708. Accordingly in one embodiment be trailing portion 708 comprises a surface 818 configured to enhance the seal formed between the leading and trailing portion 704, 708. In one embodiment the seal member 744B is in O-ring and the seal enhancing surface 818 comprises an O-ring sealing surface. As discussed above, the seal enhancing surface 818 can be configured to expand the seal member 744B by axially compression.

FIG. 20 shows one embodiment of the anchor 736 in greater detail. For example, the anchor 736 includes an engagement surface 822 that can abut against, or be brought into engagement with, a surface defined within the tunneling apparatus 700. In one embodiment, the anchor 736 is disposed in the rearward portion 802B of the lumen 802 and abuts against a rearward facing surface formed therein. The anchor 736 also has an outer periphery that is smaller than an inner size of the rearward portion 802B such that the anchor can be easily received therein. In one embodiment, the anchor 736 is a generally disk-shaped structure with a circular outer periphery 826. The anchor can be relatively thin so long as it is strong enough to withstand the forces that are applied in drawing the tunneling apparatus 700 and a percutaneous conduit through a tissue tunnel.

The anchor 736 preferably is configured to be coupled with the tension member 712 in any suitable manner. For example, FIG. 20 shows that the anchor member 736 can include a plurality of apertures 830 that can be configured to receive the tension member 712. In one embodiment, the first end 720 of the tension member 712 can be passed though both apertures 830 and secured to itself. In other embodiments a single aperture can be provided or the tension member 712 can be secured to the anchor member 736 directly. In other embodiments, a tunneling apparatus can be constructed without a separate anchor member, such as by securing the tension member 712 to the trailing portion 708.

The tunneling apparatus 700 is an advantageous way to perform at least some of the steps of the method discussed above in connection with FIG. 14. For example, prior to advancing the proximal portion of a percutaneous conduit through the tunnel 698, the tension member 712 can be advanced from the subcutaneous cavity 690 toward and through the percutaneous site 694 by any suitable means. For example a standard tunneling device or other elongate and generally stiff device can be used to advance the tension member along the tunnel 698.

In one method either before or after the tension member 712 has been so advanced, the leading portion 704 and the trailing portion 708 can be coupled with the proximal portion of a percutaneous conduit, e.g., with the proximal end 352 of the communications link 308. The coupling between the trailing portion 708 the proximal portion of the percutaneous conduit can be achieved by axially aligning the body 810 with a corresponding recess or socket in the percutaneous conduit. For example, both the body 810 and the proximal portion of the percutaneous conduit can include tri-lobular configurations whereby these end portions can be connected in any of three orientations. Of course, a multi-lobular construct can be provided, e.g., with two lobes, two or more lobes, four lobes, etc. Thereafter, the proximal portion of the percutaneous conduit can be advanced relative to the tunneling apparatus 700 such that the proximal portion of the percutaneous conduit extends over, e.g. covers, the seal structure 744. To confirm proper ceiling, the clinician can advance a proximal end of the proximal portion of the percutaneous conduit into engagement with the surface 772.

To provide enhanced engagement between the tunneling apparatus 700 and the communication link 308 or other percutaneous conduit, the seal member 744B can be radially expanded into engagement with an inner surface of the proximal end 352. Such engagement provides enhanced frictional gripping of the inner surface and provides sufficient grip to permit the tunneling apparatus 700 to be pulled through the tissue tunnel.

After the tunneling apparatus 700 has been coupled with the proximal portion of the percutaneous conduit, a force can be applied to the first end 724 of the tension member 712 outside of the percutaneous site 694 to cause the proximal portion of the percutaneous conduit to move into the tissue tunnel. Further application of force causes more of the percutaneous conduit to be drawn into the tissue tunnel. Where the percutaneous conduit comprises an isolation portion, such as the isolation portion 382 of the communications link 308, the force applied to the tension member 712 is transferred to be proximal end of the isolation portion. If the isolation portion comprises a spiral portion, further application of force to be tension member 712 causes a spiral portion to straighten, such that the spiral portion becomes low profile and can more easily pass through the tunnel 698. Further application of force to the tension member 712 draws the entire isolation portion 382 into the tissue tunnel and toward the percutaneous site 694. Further force applied to the tension member 712 causes the proximal portion of the percutaneous conduit to emerge from the exit site 694. Still further application of force to the tension member 712 and the proximal end of the percutaneous conduit causes the isolation portion to emerge from the percutaneous exit site 694.

After the isolation portion 382 of the communications link 308 (or other proximal portion of a percutaneous conduit) have emerged from the percutaneous site 694 the tunneling apparatus 700 can be disconnected from the proximal portion thereof. As discussed above, this can be accomplished by using a torque inducing tool applied to any of the flats 786.

While this description explains the inventive features of the inventions as applied to various embodiments, it will be understood that the variations in the form and details of the apparatuses or methods may be made by those of ordinary skill in the art without departing from the spirit of the inventions. The scope of the inventions is indicated by the appended claims herein, however, not by the foregoing description.

1. A percutaneous communication link for conveying signals between a extracorporeal controller and an implantable pump, the communication link comprising:
a distal end, a proximal end, and an elongate body extending therebetween, the elongate body comprising a plurality of lumens extending therethrough;
a signal wire extending through each of the lumens, the signal wires configured to convey at least one of power and control signals to the pump;
a plurality of contacts located at the proximal end for placing the communications link in electrical connection with the controller; and
a plurality of contacts located at the distal end for connecting the communications link with the pump;
wherein the lumens have a helical arrangement relative to each other to reduce electrical noise and to reduce stress on the wires.

2. The percutaneous communication link of claim 1, wherein the helical arrangement of the lumens comprises between approximately 2 and approximately 4 turns per foot.

3. The percutaneous communication link of claim 1, wherein the lumens permit the signal wires to slide longitudinally therein to enhance the flexibility of the communication link.

4. The percutaneous communication link of claim 1, further comprising a visible indicator located at least one of the proximal end and the distal end, the visible indicator indicating an order for attaching the signal wires to a pump and a controller.

5. The percutaneous communication link of claim 1, wherein the plurality of lumens comprises three lumens.

6. A communication link adapted to couple an extracorporeal system and an implantable pump for conveying information therebetween, the communication link comprising:
an implantable portion having a distal end configured to couple with the implantable pump;
an extracorporeal portion having a proximal portion configured to couple with the extracorporeal system; and
an isolation portion disposed between the implantable portion and the extracorporeal portion, the isolation portion configured to minimize the transmission to the implantable portion of at least one of movement of and forces applied to the extracorporeal portion.

7. The communication link of claim 6, wherein the isolation portion comprises a spiral arrangement adapted to be coiled in the absence of movement and forces applied to the extracorporeal portion and to become at least partially uncoiled to minimize the transmission to the implantable portion of at least one of movement of and forces applied to the extracorporeal portion.

8. The communication link of claim 7, wherein the spiral portion subtends an arc of about 540 degrees.

9. The communication link of claim 7, wherein the spiral portion can absorb at least about 2 cm of movement of the extracorporeal portion.

10. The communication link of claim 5, wherein the isolation portion has a shape memory.

11. The communication link of claim 5, wherein the isolation portion has a plurality of signal wires substantially centered therein.

12. The communication link of claim 11, further comprising a braided structure at least partially surrounding the signal wires, the braided structure being embedded in a spiral structure.

13. The communication link of claim 5, wherein the communication link further comprises a tissue ingrowth structure extending at least along a portion of the implantable portion such that when the communications link is applied to the patient the tissue ingrowth structure resides adjacent to the percutaneous site.

14.-36. (canceled)

37. A heart assist system, comprising:
an implantable pump configured to convey blood between two vascular locations;
an extracorporeal system configured to provide power and control signals to the pump; and
a communication link coupled with the extracorporeal system and with the pump for conveying information therebetween, the communication link comprising:
an implantable portion having a distal end configured to couple with the implantable pump;
an extracorporeal portion having a proximal portion configured to couple with the extracorporeal system; and
a isolation portion disposed between the implantable portion and the extracorporeal portion, the isolation portion configured to minimize the transmission of at least one of movement of and forces from the extracorporeal portion to the implantable portion.

38. The heart assist system of claim 37, further comprising a strain relief portion disposed between the implantable portion of the communication link and the implantable pump.

39. The heart assist system of claim 38, wherein the strain relief portion comprises a base portion having a plurality of arcuate channels for routing signal wires that extend through the communication link.

40. The heart assist system of claim 39, further comprising a recessed portion formed in a side surface of the implantable pump, the base portion being received in the recessed portion.

41. The heart assist system of claim 37, further comprising a tissue ingrowth member disposed along the communication link for integrating at least a portion of the communication link into the tissue, whereby a barrier to bacteria can be formed.

42.-46. (canceled)
